(19) United States
(12) Patent Application Publication
(10) Pub. No.: US 2026/0047769 A1
(43) Pub. Date: Feb. 19, 2026

(54) SYSTEMS AND DEVICES FOR BLOOD PRESSURE MEASUREMENT

(71) Applicant: Becton, Dickinson and Company, Franklin Lakes, NJ (US)

(72) Inventors: Greg Joseph Dugan, Corona, CA (US); Huy Thanh Vu, Westminster, CA (US); Max Desiré Leonard Stotijn, Hoogeveen (NL); Ilja Guelen, Ravenstein (NL); Abdulkader Sudam, Dana Point, CA (US)

(21) Appl. No.: 19/367,365

(22) Filed: Oct. 23, 2025

Related U.S. Application Data
(63) Continuation of application No. PCT/US2024/025792, filed on Apr. 23, 2024.
(60) Provisional application No. 63/497,913, filed on Apr. 24, 2023.

(51) Int. Cl. A61B 5/021; A61B 5/022

(52) U.S. Cl. CPC A61B 5/02141; A61B 5/02241; A61B 5/02108; A61B 2560/0462

(57) ABSTRACT

Systems and devices for performing hemodynamic measurements are provided. A blood pressure cuff can have a fixed tubular shape. The blood pressure cuff can include an inflatable bladder and a housing with an inflation chamber formed in between the inflatable bladder and housing. The blood pressure cuff is configured to fit onto an extremity of an individual. The inflatable bladder can include angled segments to help secure the blood pressure cuff to the extremity.

### CROSS-REFERENCE TO RELATED APPLICATION(S)

This application is a continuation of International Application No. PCT/US2024/025792, filed Apr. 23, 2024, which claims the benefit of U.S. Provisional Application No. 63/497,913, filed Apr. 24, 2023, the disclosures of which are hereby incorporated by reference in their entireties.

### TECHNOLOGICAL FIELD

The disclosure is generally directed to systems and devices for measuring blood pressure, and more specifically for systems and devices for sensing arterial pressure via a blood pressure cuff fixed in a tubular shape.

### BACKGROUND

Continuous noninvasive blood pressure monitoring systems enable real-time measurement of blood pressure waves and derived hemodynamic parameters. Generally, a noninvasive blood pressure sensor includes an arterial pressure sensor, a pressure source, a pressure regulator, and a computational system. An arterial pressure sensor can include an inflatable cuff that inflates to a pressure on the artery to be measured, and a photoplethysmograph. The photoplethysmograph can be utilized to measure parameters of the artery, such as the arterial diameter. The pressure source provides a pressure to the inflatable cuff, which is regulated by the pressure regulator. The computational system can compute and display pressure readings, and can be utilized to adjust the pressure pump, regulator, or other functions of the system.

Arterial blood pressure can be computed by knowing the pressure within the cuff and the volume of blood within the artery. One method to measure pressure is the volume clamp method, which determines blood pressure by keeping the arterial diameter constant. To perform the technique, the pressure in the cuff is adjusted to keep the diameter of the artery constant (the unloaded volume), in which the arterial diameter is determined via photoplethysmography. The pressure within the inflatable cuff that keeps the arterial diameter constant represents the arterial pressure of the artery. Generally, the technique is performed on an extremity such as a finger or a thumb. The arterial pressure waveforms of an extremity can be determined and further can be converted into other parameters such as central arterial pressure (e.g., aortic pressure) waveforms.

### SUMMARY

A system for measuring blood pressure can comprise blood pressure cuff, a source pump for providing pressure, a pressure regulator system, and a computational processing system. The blood pressure cuff can have a fixed tubular design capable of fitting and securing onto an extremity, such as a finger or thumb. The blood pressure cuff can contain an inflatable bladder and a housing. The housing can provide a port for connecting with the source pump and pressure regulator system. The housing can also provide an electrical connection for providing power to a light emitter and a light sensor, which can be a photoplethysmograph system. The electrical connection can also connect to the computational system for relaying acquired light signal data.

In some implementations, a blood pressure cuff is for use in a blood pressure measurement system. The blood pressure cuff comprises a housing and an inflatable bladder attached to the housing forming an inflation chamber therebetween. The inflatable bladder is a thin sheet of flexible molded material in a tubular shape. The housing is also fixed in a tubular shape. The inflatable bladder is contoured to conform to an extremity of an individual, for example when applied to a finger has proportionally greater width than height to accommodate the fingers anthropometric characteristic. The inflatable bladder comprises a flexible inner contact sheet, a set of two angled segments, and two tubular edges. Each angled segment is in between the flexible inner contact sheet and one of the tubular edges of the inflatable bladder.

In some implementations, each angled segment has an angle, a density, and a thickness for providing a push force, and wherein the thickness of each angled segment is greater than the thickness of the flexible inner contact sheet.

In some implementations, a vertex or radius is formed between each angled segment and the inner contact sheet. Each face independently has an angle between 90 degrees and 150 degrees to the adjoining face.

In some implementations, each angled segment has a tapered thickness such that the thickness proximal to the tubular edge is greater than the thickness proximal to the flexible inner contact sheet.

In some implementations, the flexible inner contact sheet has a thickness between 25 μm and 100 μm.

In some implementations, the inflatable bladder is a single molded sheet comprising the flexible inner contact sheet and the set of angled segments.

In some implementations, the inflatable bladder is a welded set of components comprising the flexible inner contact sheet and the set of angled segments.

In some implementations, the blood pressure cuff further comprises a set of two end caps. The inflatable bladder further comprises a flap at each tubular end. The set of end caps is utilized to secure the inflatable bladder to the housing via each flap.

In some implementations, the inflatable bladder comprises a set of two folded-over ends that each fold over an edge of the housing.

In some implementations, the inflatable bladder comprises an outer frame and foam insert for single patient one-time disposable application. The removeable bladder allows the blood pressure cuff to be used for multi-patient applications by replacing the inflatable bladder after each patient use.

In some implementations, the inflatable bladder comprises a set of one or more furrows along at least one vertex between the inner contact sheet an angled segment of the set of angled segments.

In some implementations, the housing comprises a fluid port that is in fluidic connection with the inflation chamber.

In some implementations, the housing comprises a fluid port that is in fluidic connection with the inflation chamber.

In some implementations, the blood pressure cuff further comprises a stabilizer within the inflation chamber.

In some implementations, the stabilizer is a sponge or foam fill.

In some implementations, the sponge or foam fill contains at least 50% open cells.

In some implementations, the sponge or foam fill is composed of material to yield a viscoelastic sponge or foam.

In some implementations, the sponge or foam fill has a density gradient.

In some implementations, the sponge or foam fill has greater density near the angled segments of the inflatable bladder and less density near the center the inflatable bladder.

In some implementations, the sponge or foam fill includes one or more windows to allow light signals to pass therethrough.

In some implementations, the sponge or foam fill includes one or more apertures, one or more slits, or one or more segments.

In some implementations, the stabilizer is a set of one or more springs.

In some implementations, each spring of the set is a clover-shaped ring.

In some implementations, each clover-shaped ring contacts and is secured to the housing at multiple points. Each clover-shaped ring contacts or is adjacent to the inflatable bladder at multiple points. Each clover-shaped ring includes an interconnecting portion between each contact with the housing and each contact or adjacency with the inflatable bladder. Each interconnecting portion includes a first hinge near the contact with the housing and a second hinge near the contact or adjacency with the inflatable bladder.

In some implementations, the first hinge and the second hinge of each clover-shaped ring each individually have an angle between about 30 degrees and about 150 degrees.

In some implementations, each spring of the set is an elongated ribbon that is elongated in the direction along a tubular axis of the blood pressure cuff.

In some implementations, each elongated ribbon includes a secured end that is secured to the housing, an unsecured end that is free to move in a direction along the tubular axis, and a curved portion between the secured end and the unsecured end.

In some implementations, an apex of the curved portion is in contact with or adjacent to the inflatable bladder.

In some implementations, each spring of the set is composed a material with high resiliency or high linear elasticity.

In some implementations, the stabilizer is a set of one or more columnar supports.

In some implementations, each columnar support of the set of columnar supports is in contact with a vertex adjacent to an angled segment of the set of angled segments and further in contact with and secured to the housing.

In some implementations, each columnar support of the set of columnar supports comprises a base, wherein each base provides contact is secured to the housing.

In some implementations, each columnar support of the set of columnar supports is individually provided at an angle from about 45-degrees to about 90-degrees from the housing wall to the vertex.

In some implementations, each columnar support is composed of an elastomeric material.

In some implementations, the set of one or more columnar supports and the inflatable bladder are synthesized as a single mold.

In some implementations, the blood pressure cuff further comprises a sensor system comprising light emitter and a light sensor.

In some implementations, the sensor system is a photoplethysmograph.

In some implementations, the light emitter is light emitting diode or a laser.

In some implementations, the light sensor is a photodiode.

In some implementations, the sensor system comprises at least light sensors positioned such that the sensor system can be utilized to measure blood pressure of various sizes of extremities.

In some implementations, the sensor system comprises at least two light sensors positioned such that the sensor system can be utilized to determine whether the blood pressure cuff has been fitted correctly.

In some implementations, the sensor system comprises at least two light emitters, each light emitter emitting a unique wavelength of light.

In some implementations, at least two light emitters are positioned such that the sensor system can be utilized to measure blood oxygen saturation in addition to measuring blood pressure.

In some implementations, the sensor system is within the inflation chamber and the inflatable bladder contains a translucent window to allow light to pass therethrough.

In some implementations, the sensor system is within the inflation chamber and the inflatable bladder is translucent to allow light to pass therethrough.

In some implementations, the sensor system is on the flexible inner contact sheet of the inflatable bladder.

In some implementations, a blood pressure monitoring system comprises a blood pressure cuff comprising a housing and an inflatable bladder attached the housing forming an inflation chamber therebetween. The housing and the inflatable bladder are fixed in a tubular shape. The housing comprises a fluid port in fluidic connection with the inflation chamber and a photoplethysmograph system. The blood pressure monitoring system further comprises a pressure source in fluidic connection with the inflation chamber via the fluid port. The blood pressure monitoring system further comprises a pressure regulatory system provided in between the pressure source and the blood pressure cuff. The blood pressure monitoring system further comprises a computational processing system in communication with the photoplethysmograph system and the pressure regulatory system.

In some implementations, the inflatable bladder comprises a flexible inner contact sheet and a set of two angled segments. Each angled segment is in between the flexible inner contact sheet and one of the tubular edges of the inflatable bladder.

In some implementations, each angled segment has an angle, a density and a thickness capable of providing a push force. The thickness of each angled segment is greater than the thickness of the flexible inner contact sheet.

In some implementations, a vertex is formed between each angled segment and the inner contact sheet. Each vertex independently has an angle between 90 degrees and 150 degrees.

In some implementations, each angled segment has a tapered thickness such that the thickness proximal to the tubular edge is greater than the thickness proximal to the flexible inner contact sheet.

In some implementations, the flexible inner contact sheet has a thickness between 25 μm and 100 μm.

In some implementations, the blood pressure monitoring system further comprises a stabilizer within the inflation chamber.

In some implementations, the stabilizer is a sponge or foam fill.

In some implementations, the sponge or foam fill contains at least 50% open cells.

In some implementations, the sponge or foam fill is composed of material to yield a viscoelastic sponge or foam.

In some implementations, the sponge or foam fill has a density gradient.

In some implementations, the sponge or foam fill has greater density near the angled segments of the inflatable bladder and less density near the center the inflatable bladder.

In some implementations, the sponge or foam fill includes one or more windows to allow light signals to pass therethrough.

In some implementations, the sponge or foam fill includes one or more apertures, one or more slits, or one or more segments.

In some implementations, the stabilizer is a set of one or more springs.

In some implementations, each spring of the set is a clover-shaped ring.

In some implementations, each clover-shaped ring contacts and is secured to the housing at multiple points. Each clover-shaped ring contacts or is adjacent to the inflatable bladder at multiple points. Each clover-shaped ring includes an interconnecting portion between each contact with the housing and each contact or adjacency with the inflatable bladder. Each interconnecting portion includes a first hinge near the contact with the housing and a second hinge near the contact or adjacency with the inflatable bladder.

In some implementations, the first hinge and the second hinge each individually have an angle between about 30 degrees and about 150 degrees.

In some implementations, each spring of the set is an elongated ribbon that is elongated in the direction along a tubular axis of the blood pressure cuff.

In some implementations, each elongated ribbon includes a secured end that is secured to the housing, an unsecured end that is free to move in a direction along the tubular axis, and a curved portion between the secured end and the unsecured end.

In some implementations, an apex of the curved portion is in contact with or adjacent to the inflatable bladder.

In some implementations, each spring of the set is composed a material with high resiliency or high linear elasticity.

In some implementations, the stabilizer is a set of one or more columnar supports.

In some implementations, each columnar support of the set of columnar supports is in contact with a vertex adjacent to an angled segment of the set of angled segments and further in contact with and secured to the housing.

In some implementations, each columnar support of the set of columnar supports comprises a base, wherein each base provides contact is secured to the housing.

In some implementations, each columnar support is composed of an elastomeric material.

In some implementations, each columnar support of the set of columnar supports is individually provided at an angle from about 45-degrees to about 90-degrees from the housing wall to the vertex.

In some implementations, the set of one or more columnar supports and inflatable bladder are synthesized as a single mold.

In some implementations, the pressure regulatory system is capable of sensing and regulating the amount of pressure being provided to the inflation chamber via the pressure source.

In some implementations, the photoplethysmograph system is capable of transmitting arterial diameter data to the computational system.

In some implementations, a molded bladder comprises a sheet of molding fixed in a tubular shape having an inner wall, an outer wall, a set of two angled segments, and a contact sheet along the inner wall in between the two angled segments. A first angled segment bends from the inner wall and angles outward to a first tubular end and a second angled segment bends from the inner wall and angles outward to a second tubular end. The sheet of molding comprises a means for securement to a housing having a tubular shape such that when the sheet of molding is secured to the housing, the sheet of molding and housing are capable of forming a sealed chamber there in-between.

In some implementations, each angled segment has an angle and a thickness for providing a push force. The

In some implementations, thickness of each angled segment is greater than the thickness of the flexible inner contact sheet.

In some implementations, a vertex is formed between each angled segment and the inner contact sheet. Each vertex independently has an angle between 90 degrees and 150 degrees.

In some implementations, each angled segment has a tapered thickness such that the thickness proximal to the tubular edge is greater than the thickness proximal to the flexible inner contact sheet.

In some implementations, the flexible inner contact sheet has a thickness between 25 μm and 100 μm.

In some implementations, the means for securement is a flap that at each tubular end. Each flap is configured to provide securement via a set of two end caps that are configured to secure the inflatable bladder to the housing via each flap.

In some implementations, the inner wall, first tubular end, and second tubular end are concentric.

In some implementations, a molded bladder further comprises comprising a set of one or more columnar supports.

In some implementations, each columnar support of the set of columnar supports is in contact with a vertex adjacent to an angled segment of the set of angled segments and extends outwardly away from the vertex.

In some implementations, each columnar support of the set of columnar supports is comprises a base. Each base provides a means of securement to the housing.

In some implementations, each columnar support of the set of columnar supports is individually provided at an angle from about 45-degrees to about 90-degrees from the housing wall to the vertex.

In some implementations, the set of one or more columnar supports and the inflatable bladder are synthesized as a single mold.

In some implementations, the sheet of molding fixed in a tubular shape is composed of an elastomeric material.

In some implementations, a set of components is for assembly of a blood pressure cuff. The set of components comprises a housing fixed in a tubular shape. The set of components further comprises a sheet of molding fixed in a tubular shape having an inner wall, an outer wall, a set of two angled segments, and a contact sheet along the inner wall in between the two angled segments. A first angled segment bends from the inner wall and angles outward to a first tubular end and a second angled segment bends from the inner wall and angles outward to a second tubular end. The sheet of molding comprises a flap at each tubular end. The set of components further comprises a set of two end caps. The set of end caps is configured to secure the inflatable bladder to the housing via each flap such that a sealed chamber is formed between the inflatable bladder and the housing when assembled.

In some implementations, each angled segment has an angle, a density, and a thickness for providing a push force. The thickness of each angled segment is greater than the thickness of the flexible inner contact sheet.

In some implementations, each a vertex is formed between each angled segment and the inner contact sheet. Each vertex independently has an angle between 90 degrees and 150 degrees.

In some implementations, each angled segment has a tapered thickness such that the thickness proximal to the tubular edge is greater than the thickness proximal to the flexible inner contact sheet.

In some implementations, the flexible inner contact sheet has a thickness between 25 μm and 100 μm.

In some implementations, the housing comprises a fluid port that is configured such that it is in fluidic connection with the inflation chamber when assembled.

In some implementations, the inner wall, first tubular end, and second tubular end are configured to be concentric when assembled.

In some implementations, the sheet of molding fixed in a tubular shape is composed of an elastomeric material.

In some implementations, the set of components further comprises a stabilizer. The stabilizer is configured to fit within the inflation chamber when assembled.

In some implementations, the stabilizer is a sponge or foam fill.

In some implementations, the stabilizer is a set of one or more springs.

In some implementations, each spring of the set is a clover-shaped ring.

In some implementations, each spring of the set is an elongated ribbon that is elongated in the direction along a tubular axis of the blood pressure cuff when assembled.

In some implementations, the stabilizer is a set of one or more columnar supports.

In some implementations, the set of one or more columnar supports and the inflatable bladder are synthesized as a single mold.

In some implementations, the set of components further comprises a sensor system comprising light emitter and a light sensor configured to be installed within the housing or within the inflatable bladder.

In some implementations, the sensor system is a photoplethysmograph.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1 provides a system for performing blood pressure measurements.

FIGS. 2A to 2C provide an example of blood pressure cuff having a molded bladder with sensor installed in the housing.

FIGS. 2D to 2F provide an example of blood pressure cuff having a molded bladder with sensors installed on the bladder.

FIGS. 3A to 3C provide an example of blood pressure cuff having a welded bladder.

FIGS. 4A to 4C provide an example of blood pressure cuff having an enveloped bladder.

FIGS. 5A and 5B provide an example of an inflatable bladder having a scalloped design.

FIGS. 5C and 5D provides an example of an inflatable bladder configured to insert within a housing.

FIGS. 5E to 5G provide an example of installing an inflatable bladder within a housing.

FIGS. 6A to 6G provide examples of a sponge or foam fill stabilizer.

FIGS. 7A and 7B provide an example of a set of clover shaped spring stabilizers.

FIGS. 8A and 8B provide an example of a set of elongated ribbon spring stabilizers.

FIGS. 9A and 9B provide an example of a set of columnar support stabilizers.

FIGS. 10A to 10D provide examples of positioning light emitters and light sensors.

FIG. 11 provides a method of using a blood pressure cuff.

FIG. 12 provides a conceptual illustration of a computational processing system for performing blood pressure monitoring.

### DETAILED DESCRIPTION

The current disclosure details systems and devices for performing blood pressure measurements of an artery. Generally, the systems and devices include a blood pressure cuff fixed in a tubular shape, the cuff comprising a flexible inflatable bladder, a housing, and an inflation chamber therebetween. Inflation of the inflatable bladder provides pressure onto a body extremity to alter the diameter the extremity's artery. The blood pressure cuff further includes a light emitter and a light sensor for performing photoplethysmography. The blood pressure cuff can be utilized within continuous blood pressure monitoring system. The blood pressure monitoring system can monitor the arterial diameter and adjust the pressure within inflatable bladder such that an arterial waveform of the extremity can be measured and generated. The arterial waveform of the extremity can be utilized to compute various downstream parameters, such as (for example) a more central arterial waveform.

A blood pressure cuff can be utilized on any extremity capable of receiving the cuff that is fixed in a tubular shape. Thus, the cuff can be specifically sized to fit onto a finger, a thumb, a forearm, an upper arm, a toe, among other extremities. In many instances, the cuff is sized for a digit of the hand, such as a finger or a thumb. The cuff can continually measure the arterial pressure within the extremity to generate an arterial pressure waveform of the extremity. To enhance the usefulness of the measured arterial pressure, the measured waveform can be transformed into a more central arterial pressure, such as (for example) an aortic pressure.

Blood pressure cuffs of the prior art generally were designed as a flat sheet that can roll over and encircle an extremity such that the sheet formed a circle around the extremity. Typically, Velcro, tape, or another adjustable clasp is utilized to hold the cuff in a tubular shape around the extremity, allowing for easy sizing and tightening of the pressure cuff. This design, however, has a variety of issues that arise when the cuff is applied too tight or too loose, resulting in improper pressure readings. Accordingly, continuous blood monitoring systems can benefit from a cuff that does not require encircling and tightening around the extremity.

Here, novel systems and devices are directed to pressurizable cuffs that are fixed in a permanent tubular shape and are capable of being slid onto the extremity. To ensure appropriate tightness around the extremity, the inflatable bladder is designed with appropriate push forces to snugly and securely fit onto the extremity yet still maintain an ability to inflate to pressure to yield an unloaded volume. The inflatable bladder can be constructed of a semirigid, yet elastomeric and flexible material that can secure an extremity and respond to pressure dynamics. The inflatable bladder can have a radial inner contact sheet that is elastomeric and flexible sheet of material configured to conform to and be in contact with the extremity. The flexible contact sheet can be a thin profile to allow the sheet to expand outward upon inflation and contract inward upon deflation in accordance with changes of pressure within an inflation chamber. Two radial angled surfaces can extend from the flexible inner contact sheet at the tubular ends, forming two radial vertices between the angled surfaces and inner contact sheet. The angled surfaces can be angled towards and secured to an outer housing, which can be rigid or semirigid housing. An inflation chamber is formed within the space between the outer housing and the inflatable bladder. Each of the two angled surfaces can have a thickness and/or density to provide a radially inward push force normal to the inner contact sheet such that when an extremity is inserted within the cuff and applies a radially outward pressure on the bladder, the radially inward normal pressure snugly secures the extremity. Stabilizers can also be incorporated with the inflatable bladder to provide further strengthen push forces to help secure the extremity.

The described systems, devices, and methods should not be construed as limiting in any way. Instead, the present disclosure is directed toward all novel and nonobvious features and aspects of the various disclosed systems and devices, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various examples of blood pressure monitoring systems and blood pressure cuffs are disclosed herein, and any combination of these examples can be made unless specifically excluded. For example, any of the inflatable bladders disclosed, can be used with any type of blood pressure cuff housing, even if a specific combination is not explicitly described. Likewise, the different constructions and features of blood pressure monitoring systems and blood pressure cuffs can be mixed and matched, such as by combining any inflatable bladder design, any blood pressure cuff housing, any system of light emitters and sensors etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

### Systems and Devices for Blood Pressure Monitoring

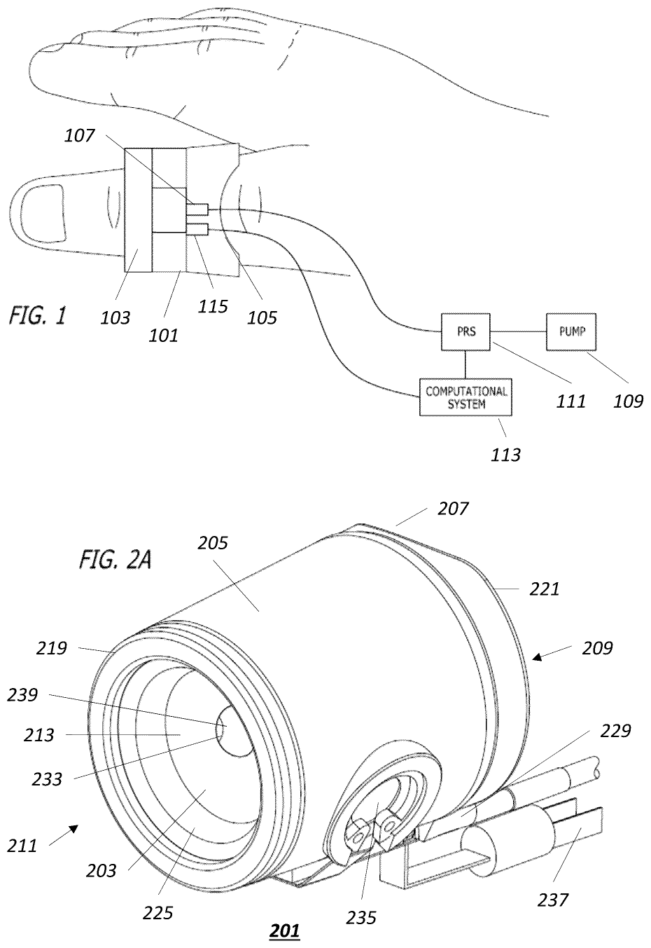

The various blood pressure cuffs of the current disclosure can be utilized within a noninvasive blood pressure monitoring system. Generally, the noninvasive blood pressure monitoring system includes a photoplethysmograph, a pressure source, a pressure regulator, and a computational system. Provided in FIG. 1 is an example of a noninvasive blood monitoring system as would be utilized on the thumb of an individual. On the thumb is a blood pressure cuff 101 with a fixed tubular shape. Blood pressure cuff 101 includes an elastomeric inflatable bladder in contact with the thumb, a more rigid housing 103, and an inflation chamber therebetween. Blood pressure cuff 101 incorporates a knuckle contour 105 that is complimentary to the shape of a knuckle of the thumb and assist with alignment of the light sensors over the proper palmar digital arteries along underside of finger. Although blood pressure cuff 101 is shown to be positioned on the thumb, it is to be understood that the cuff can be sized for various extremities, including a finger, a forearm, an upper arm, a toe or any other extremity of the body.

Housing 103 incorporates a fluid port 107 in fluidic connection with a pump 109 to provide pressure to the inflation chamber. Any fluid port can be utilized, such as (for example) a nozzle, valve, or gasket. A pressure regulator system 111 can be provided in between blood pressure cuff 101 and pump 109, which can sense and/or regulate the amount of pressure being provided to the inflation chamber. Pressure regulator system 111 can be controlled by a computational system 113, which can provide instructions to the regulator system to control the pressure in accordance with a computational application for measuring and monitoring blood pressure.

Housing 103 further incorporates an electrical connection 115 that can transmit power and/or data to and from the blood pressure cuff 101. Electrical connection 115 can provide power and data for a light emitter and a light sensor, which are provided within housing 103. The light emitter and light sensor can work in concert to measure the diameter of the artery within the thumb such that an accurate arterial blood pressure reading can be determined. The data collected by the light emitter and light sensor can be provided to the computational system 113 via electrical connection 115, which in turn can work in concert with pressure regulator system 111 to provide pressure for controlling the arterial diameter.

Computational system 113 can incorporate various computational programs for computing various arterial pressures (e.g., aortic pressure) from the measured arterial pressure within the thumb. Computational system 113 can further include a screen and user interface to display the various measured and computed arterial pressures and allow a user to interact with the blood pressure system.

While a specific blood monitoring system configuration is described above with reference to FIG. 1, it should be readily appreciated that various blood monitoring systems and/or other medical monitoring utilized in the provision of blood pressure monitoring can be implemented in a variety of configurations. Accordingly, the various blood pressure cuffs described herein should be understood as not to be limited to any specific blood monitoring system configuration, but instead can be implemented using any variety of blood monitoring or medical monitoring systems capable measuring arterial blood pressure.

Various pressurizable cuff systems of the current disclosure have a permanently fixed tubular shape and incorporate a semi-rigid yet elastomeric inflatable bladder attached to a rigid or semirigid housing. In many implementations, the housing of the cuff system has a greater stiffness than the inflatable bladder, which ensures that when the bladder is inflated, the housing does not deform in response to the pressure. Several materials and/or polymers are known to be elastomeric and can be used. An inflatable can comprise (but is not limited to) one or more of the following materials: polytetrafluoroethylene (PTFE), polypropylene (PP), ethylene tetrafluoroethylene (ETFE), polyethylene (PE), high density polyethylene (HDPE), polyethylene terephthalate (PET), polyvinylchloride (PVC), polystyrene (PS), polyurethane (PU), ethylene propylene diene monomer (EPDM), isobutylene isoprene, neoprene, rubber, nitrile, latex, and polysiloxane. Several polymers are known to be rigid or semirigid and can be used as the housing, including (but not limited to) liquid crystal polymer (LCP), polyetheretherketone (PEEK), polyetherimide (PEI), polylactide (PLA), polypropylene (PP), polyphthalamide (PPA), polyphenylene sulfide (PPS), polystyrene (PS), and polyvinyl chloride (PVC). Further, as is well appreciated in the art, the elasticity and stiffness of various material and polymers can be altered by chemical modification, incorporating reinforced components, blending various materials, or altering density. For instance, many polymers can be further stiffened by incorporating glass fiber.

A pressurizable cuff can incorporate various bladder designs. In some implementations, the bladder is a single molded component that is secured to a housing. In some implementations, a bladder comprises welded components. In some implementations, a bladder envelops onto a portion of the housing. In some implementations, the light emitter and light sensor are installed on the housing and at least a portion of the inflatable bladder is is translucent to allow light travel from the emitter through the bladder to the extremity and back through the bladder to the light sensor. In some instances, the light emitter and light sensor are on installed on the surface of the inflatable bladder.

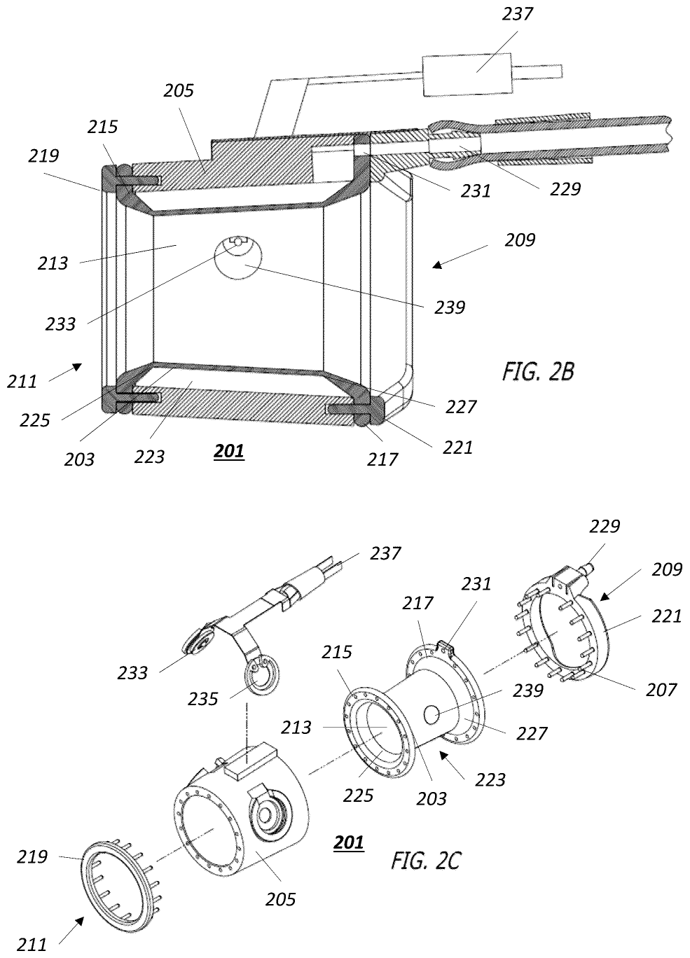

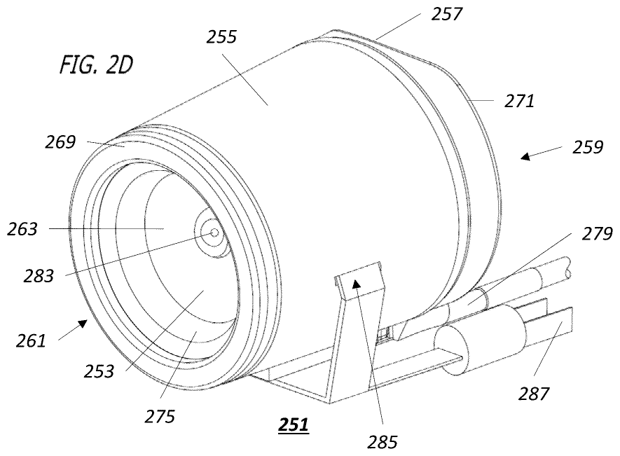

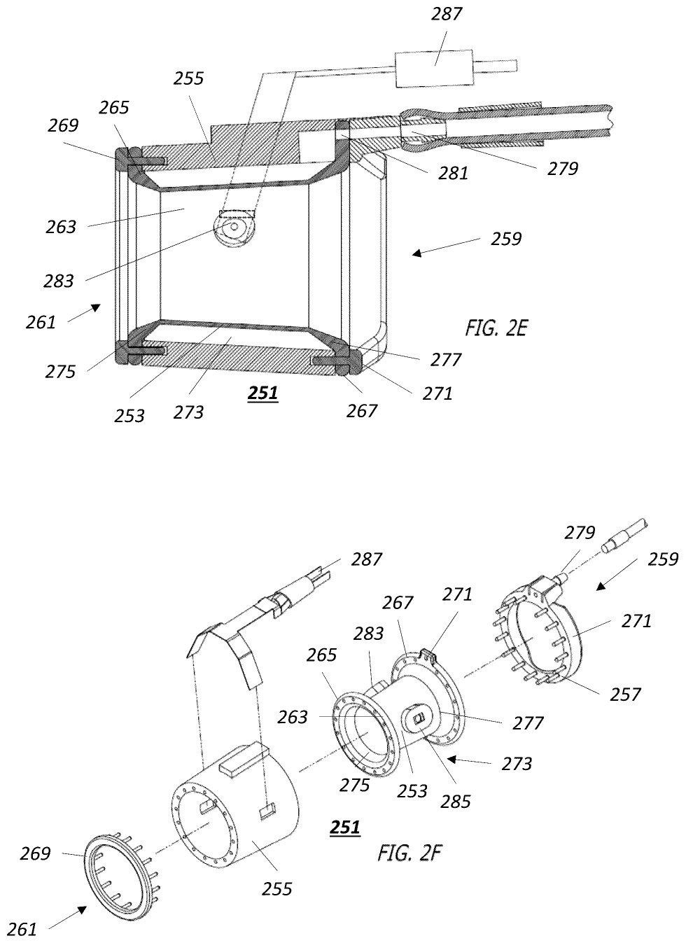

Provided in FIGS. 2A to 2C is a blood pressure cuff 201 that incorporates a single component molded inflatable bladder 203 secured to housing 205. Blood pressure cuff 201 has a tubular shape and is generally constructed to tightly fit onto an extremity. As shown, blood pressure cuff 201 is configured to fit a thumb and includes a knuckle contour 207 that assists in alignment of the sensor system over the palmar digital arteries to be utilized for hemodynamic measurement. A first tubular end 209 can have a larger diameter than the opposite tubular end 211 to accommodate the contours of the thumb. It is to be understood that the tubular design can be adapted to fit the contours of any extremity. Further, to accommodate a variety of extremity sizes, blood pressure cuff 201 can be constructed in variety sizes. For instance, blood pressure cuff 201 can be constructed having a small size, a medium size, and a large size, where the size is correlated with the diameter of the tubular shape.

Inflatable bladder 203 has a tubular shape with a flexible contact sheet 213 that provides contact to the thumb. Inflatable bladder 203 is molded to incorporate a first edge 215 and second edge 217 for securing to housing 205 via cap end 219 and cap end 221, respectively. As shown, cap end 219 and cap end 221 utilize a gasket-like connection to provide a sealed inflation chamber 223 in between inflatable bladder 203 and housing 205. It should be understood, however, that any connection for providing a sealed inflation chamber can be utilized.

In between edge 215 and flexible contact sheet 213 is angled segment 225 and in between edge 217 and flexible contact sheet 213 is angled segment 227, forming vertices between the face of the inner contact sheet and the face of each angled segment. Angled segments 225 and 227 each have an angle, a thickness, and density to provide a radially inward push force normal to flexible contact sheet 213 such that blood pressure cuff is secure when fitted on the thumb. Greater thickness of angled segments also eliminates pressure fluctuations while measuring blood pressure to ensure stable measurement. The push forces also ensure flexible contact sheet 213 has sufficient contact with the thumb when fitted therein. The angle of each vertex between the face of contact sheet 213 and the face of angled segments 225 and 227 can vary, but can generally be between 90 degrees and 150 degrees. The closer the angle is to 90 degrees, the more push force the angled segment can provide. In various implementations, the angle of the vertex between flexible contact sheet 213 and angle segment 225 or between angled segment 227 is between about 80 degrees and about 100 degrees, between about 90 degrees and about 110 degrees, between about 100 degrees and about 120 degrees, between about 110 degrees and about 130 degrees, between about 120 degrees and about 140 degrees, between about 130 degrees and about 150 degrees, or between about 140 degrees and about 160 degrees.

Flexible contact sheet 213 has a thickness that allows elasticity and flexibility such that the inner contact sheet contours to the dimensions of the thumb when inserted within and further to allow expansion and contraction in accordance with inflation and deflation of inflation chamber 223. The thickness of flexible contact sheet 213, angled segment 225, and angled segment 227 depend on the inflatable bladder material and its elasticity, density and/or stiffness. In various instances, the thickness of flexible contact sheet 213 is between 25 μm and 100 μm. In various instances, the thickness of flexible contact sheet 213 is between about 20 μm and about 40 μm, between about 30 μm and about 50 μm, between about 40 μm and about 60 μm, between about 50 μm and about 70 μm, between about 60 μm and about 80 μm, between about 70 μm and about 90 μm, or between about 80 μm and about 100 μm. Angled segment 225 and angled segment 227 can have a tapered thickness such that the thickness adjacent to edges 215 and 217 is greater than the thickness adjacent to the inner contact sheet.

Inflation chamber 223 is in fluidic connection with port 229, which is in connection to the pressure supply pump and pressure regulator system. Bladder edge 215 can incorporate an aperture 231 to allow pressure to flow from port 229 into inflation chamber 223. Although port 229 is shown to be located in proximity to tubular end 209, it can be positioned anywhere along the housing as long as it provides a fluidic connection to inflation chamber 223. Accordingly, in various instances, port 229 is proximal to tubular end 209, port 229 is proximal to tubular end 211, or port 229 is located more centrally between tubular end 209 and tubular end 211.

Blood pressure cuff 201 further incorporates a sensor system comprising a light emitter 233 and a light sensor 235 for sensing arterial diameter or performing other tasks that would be useful in the provision of hemodynamic monitoring (e.g., measuring blood oxygen levels). Light emitter 233 and light sensor 235 are in connection with an electrical connector 237 such that power and data can be transmitted to and from the light emitter and light sensor. As shown, light emitter 233 and light sensor 235 are secured within housing 205. When light emitter 233 and light sensor 235 are secured within housing 205, inflatable bladder 203 is to be translucent to the emitted light. Inflatable bladder 203 can incorporate a translucent window 239 or be translucent throughout. Light emitter 233 can be any light source for providing the requisite light wavelength utilized for measuring the arterial diameter, including (but not limited to) a light emitting diode or laser. In some instances, the light emitter can include filter (e.g., narrow band filter) for filtering the provided light wavelength. Likewise, light sensor 235 can be any sensor for detecting light signals, including (but not limited to) a photodiode. In some instances, the system of light emitter 233 and light sensor 235 is a photoplethysmograph.

Provided in FIGS. 2D to 2F is a blood pressure cuff 251 that incorporates a single component molded inflatable bladder 253 secured to housing 255 with light emitter 283 and a light sensor 285 on inner contact sheet 263 of the inflatable bladder. Blood pressure cuff 251 has a tubular shape and is generally constructed to tightly fit onto an extremity. As shown, blood pressure cuff 251 is configured to fit a thumb and includes a knuckle contour 257 that assists in alignment of the sensor system over the palmar digital arteries to be utilized for hemodynamic measurement. A first tubular end 259 can have a larger diameter than the opposite tubular end 261 to accommodate the contours of the thumb. It is to be understood that the tubular design can be adapted to fit the contours of any extremity. Further, to accommodate a variety of extremity sizes, blood pressure cuff 251 can be constructed in variety sizes. For instance, blood pressure cuff 251 can be generated having a small size, a medium size, and a large size, where the size is correlated with the diameter of the tubular shape.

Inflatable bladder 253 has a tubular shape with a flexible contact sheet 263 that provides contact to the thumb. Inflatable bladder 253 is molded to incorporate a first edge 265 and second edge 267 for securing to housing 255 via cap end 269 and cap end 271, respectively. As shown, cap end 269 and cap end 271 utilize a gasket-like connection to provide a sealed inflation chamber 273 in between inflatable bladder 253 and housing 205. It should be understood, however, that any connection for providing a sealed inflation chamber can be utilized.

In between edge 265 and flexible contact sheet 263 is angled segment 275 and in between edge 267 and flexible contact sheet 263 is angled segment 277, forming vertices between the face of the inner contact sheet and the face of each angled segment. Angled segments 275 and 277 each have an angle, a thickness, and density to provide a radially inward push force normal to contact sheet 263 such that blood pressure cuff is secure when fitted on the thumb. Greater thickness of the angled segments also eliminates pressure fluctuations while measuring blood pressure to ensure stable measurement. The push forces also ensure flexible contact sheet 263 has sufficient contact with the thumb when fitted therein. The angle of each vertex between the face of contact sheet 263 and the face of angled segments 275 and 277 can vary, but can generally be between 90 degrees and 150 degrees. The closer the angle is to 90 degrees, the more push force the angled segment can provide. In various implementations, the angle of the vertex between flexible contact sheet 263 and angled segment 275 or between angled segment 277 is between about 80 degrees and about 100 degrees, between about 90 degrees and about 110 degrees, between about 100 degrees and about 120 degrees, between about 110 degrees and about 130 degrees, between about 120 degrees and about 140 degrees, between about 130 degrees and about 150 degrees, or between about 140 degrees and about 160 degrees.

Flexible contact sheet 263 has a thickness that allows elasticity and flexibility such that the inner contact sheet contours to the dimensions of the thumb when inserted within and further to allow expansion and contraction in accordance with inflation and deflation of inflation chamber 273. The thickness of contact sheet 263, angled segment 275, and angled segment 277 depend on the inflatable bladder material and its elasticity, density and/or stiffness. In various instances, the thickness of contact sheet 263 is between 25 μm and 100 μm. In various instances, the thickness of flexible contact sheet 263 is between about 20 μm and about 40 μm, between about 30 μm and about 50 μm, between about 40 μm and about 60 μm, between about 50 μm and about 70 μm, between about 60 μm and about 80 μm, between about 70 μm and about 90 μm, or between about 80 μm and about 100 μm. Angled segment 275 and angled segment 277 can have a tapered thickness such that the thickness adjacent to edges 265 and 267 is greater than the thickness adjacent to the inner contact sheet.

Inflation chamber 273 is in fluidic connection with port 279, which is in connection to the pressure supply pump and pressure regulator system. Bladder edge 265 can incorporate an aperture 281 to allow pressure to flow from port 279 into inflation chamber 273. Although port 279 is shown to be located in proximity to tubular end 259, it can be positioned anywhere along the housing as long as it provides a fluidic connection to inflation chamber 273. Accordingly, in various instances, port 279 is proximal to tubular end 259, port 279 is proximal to tubular end 261, or port 279 is located more centrally between tubular end 259 and tubular end 261.

Blood pressure cuff 251 further incorporates a sensor system comprising a light emitter 283 and a light sensor 285 for sensing arterial diameter or performing other tasks that would be useful in the provision of hemodynamic monitoring (e.g., measuring blood oxygen levels). Light emitter 283 and light sensor 285 are in connection with an electrical connector 287 such that power and data can be transmitted to and from the light emitter and light sensor. As shown, light emitter 283 and light sensor 285 are secured on inner contact sheet 263 of inner bladder 253. Light emitter 283 can be any light source for providing the requisite light wavelength utilized for measuring the arterial diameter, including (but not limited to) a light emitting diode or laser. In some instances, the light emitter can include filter (e.g., narrow band filter) for filtering the provided light wavelength. Likewise, light sensor 285 can be any sensor for detecting light signals, including (but not limited to) a photodiode. In some instances, the system of light emitter 283 and light sensor 285 is a photoplethysmograph.

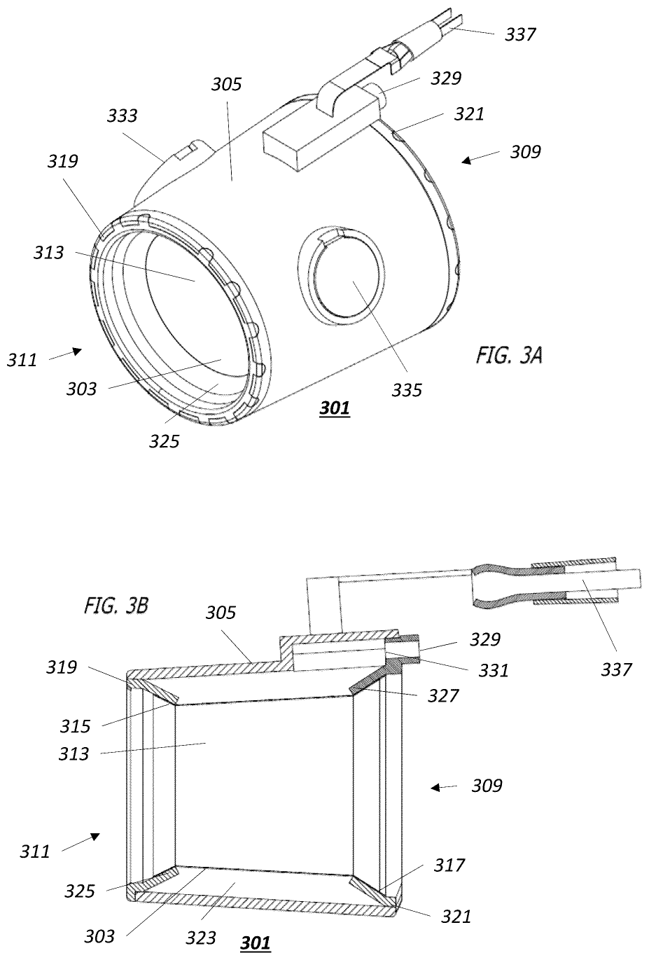

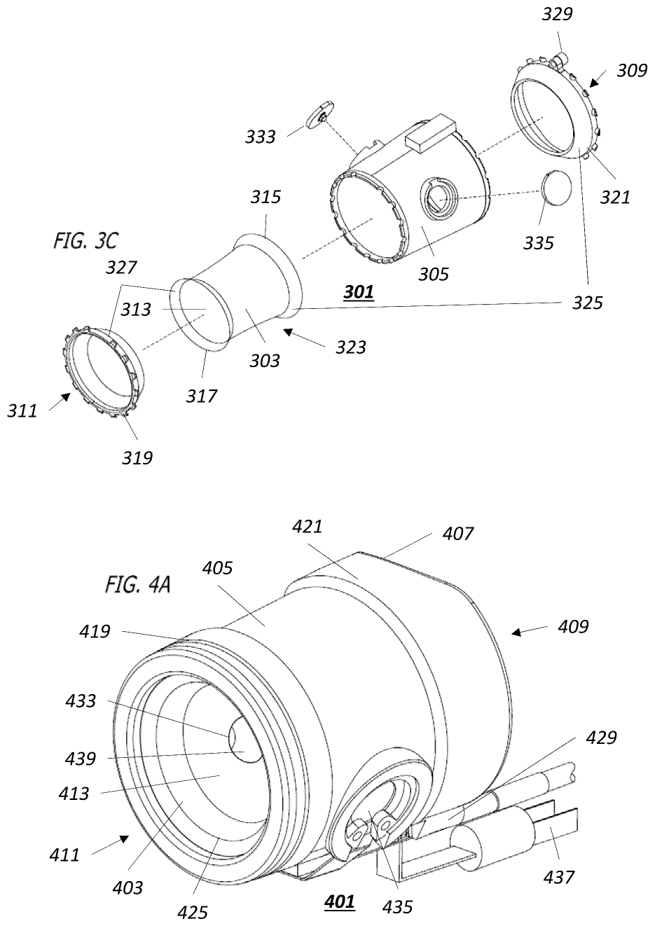

Provided in FIGS. 3A to 3C is a blood pressure cuff 301 that incorporates a welded inflatable bladder 303 secured to housing 305 via welding to end cap 319 and end cap 321 to form a welded bladder. Blood pressure cuff 301 has a tubular shape and is generally constructed to tightly fit onto an extremity. As shown, blood pressure cuff 301 is configured to fit a thumb. A first tubular end 309 can have a larger diameter than the opposite tubular end 311 to accommodate the contours of the thumb. It is to be understood that the tubular design can be adapted to fit any extremity. Further, to accommodate a variety of extremity sizes, blood pressure cuff 301 can be constructed in variety sizes. For instance, blood pressure cuff 301 can be generated having a small size, a medium size, and a large size, where the size is correlated with the diameter of the tubular shape.

Inflatable bladder 303 has a tubular shape with a flexible contact sheet 313 that provides contact to the thumb. Welding of inflatable bladder 303 to end cap 319 and to end cap 321 allows the bladder to be secured to a flap of first edge 315 and a flap of second edge 317, which is allows for securing to housing 305. End cap 319 and end cap 321 can be composed of a semi-rigid yet elastic material such that it can each be welded to inflatable bladder 303 prior to securement to the housing, which allows the end caps to deform to fit through the internal space within housing 305. Alternatively, end cap 319 can be securing to inflatable bladder 303, then passed through internal space within housing 305 prior to end cap 321 to housing 305, or vice versa. In any case, welding and securement of inflatable bladder 303 to housing 305 yields an inflation chamber 323 therebetween. It should be understood, however, that any connection for providing a sealed inflation chamber can be utilized.

In between edge 315 and flexible contact sheet 313 is angled segment 225 and in between edge 317 and flexible contact sheet 313 is angled segment 227, forming vertices between the face of contact sheet and the face of each angled segment. Angled segments 325 and 327 are formulated from the welding of end caps 319 and 321 with inflatable bladder 303. Angled segments 325 and 327 each have an angle, a thickness, and density to provide a radially inward push force normal to contact sheet 313 such that blood pressure cuff is secure when fitted on the thumb. Greater thickness of the angled segments also eliminates pressure fluctuations while measuring blood pressure to ensure stable measurement. The push forces also ensure contact sheet 313 has sufficient contact with the thumb when fitted therein. The angle of each vertex between the face of contact sheet 313 and face of angled segments 325 and 327 can vary, but can generally be between 90 degrees and 150 degrees. The closer the angle is to 90 degrees, the more push force the angled segment can provide. In various implementations, the angle of the vertex between contact sheet 313 and angled segment 325 or between angled segment 327 between about 80 degrees and about 100 degrees, between about 90 degrees and about 110 degrees, between about 100 degrees and about 120 degrees, between about 110 degrees and about 130 degrees, between about 120 degrees and about 140 degrees, between about 130 degrees and about 150 degrees, or between about 140 degrees and about 160 degrees.

Flexible contact sheet 313 has a thickness that allows elasticity and flexibility such that the inner contact sheet contours to the dimensions of the thumb when inserted within and further to allow expansion and contraction in accordance with inflation and deflation of inflation chamber 323. The thickness of flexible contact sheet 313, angled segment 325, and angled segment 327 depend on the inflatable bladder material and its elasticity, density and/or stiffness. In various instances, the thickness of flexible contact sheet 313 is between 25 μm and 100 μm. In various instances, the thickness of contact sheet 313 is between about 20 μm and about 40 μm, between about 30 μm and about 50 μm, between about 40 μm and about 60 μm, between about 50 μm and about 70 μm, between about 60 μm and about 80 μm, between about 70 μm and about 90 μm, or between about 80 μm and about 100 μm. Angled segment 325 and angled segment 327 can have a tapered thickness such that the thickness adjacent to edges 315 and 317 is greater than the thickness adjacent to the inner contact sheet.

Inflation chamber 323 is in fluidic connection with port 329, which is in connection to the pressure supply pump and pressure regulator system. End cap 319 can incorporate an aperture 331 to allow pressure to flow from port 329 into inflation chamber 323. Although port 329 is shown to be located in proximity to tubular end 309, it can be positioned anywhere along the housing as long as it provides a fluidic connection to inflatable chamber 323. Accordingly, in various instances, port 329 is proximal to tubular end 309, port 329 is proximal to tubular end 311, or port 329 is located more centrally between tubular end 309 and tubular end 311.

Blood pressure cuff 301 further incorporates a sensor system comprising a light emitter 333 and a light sensor 335 for sensing arterial diameter or performing other tasks that would be useful in the provision of hemodynamic monitoring (e.g., measuring blood oxygen levels). Light emitter 333 and light sensor 335 are in connection with an electrical connector 337 such that power and data can be transmitted to and from the light emitter and light sensor. As shown, light emitter 333 and light sensor 335 are secured within housing 305, however, the light emitter and light sensor can be provided on the surface of flexible contact sheet 313. When light emitter 333 and light sensor 335 are secured within housing 305, inflatable bladder 303 is to be translucent to the emitted light. Inflatable bladder 303 can incorporate a translucent window or be translucent throughout. Light emitter 333 can be any light source for providing the requisite light wavelength utilized for measuring the arterial diameter, including (but not limited to) a light emitting diode or laser. In some instances, the light emitter can include filter (e.g., narrow band filter) for filtering the provided light wavelength. Likewise, light sensor 335 can be any sensor for detecting light signals, including (but not limited to) a photodiode. In some instances, the system of light emitter 333 and light sensor 335 is a photoplethysmograph.

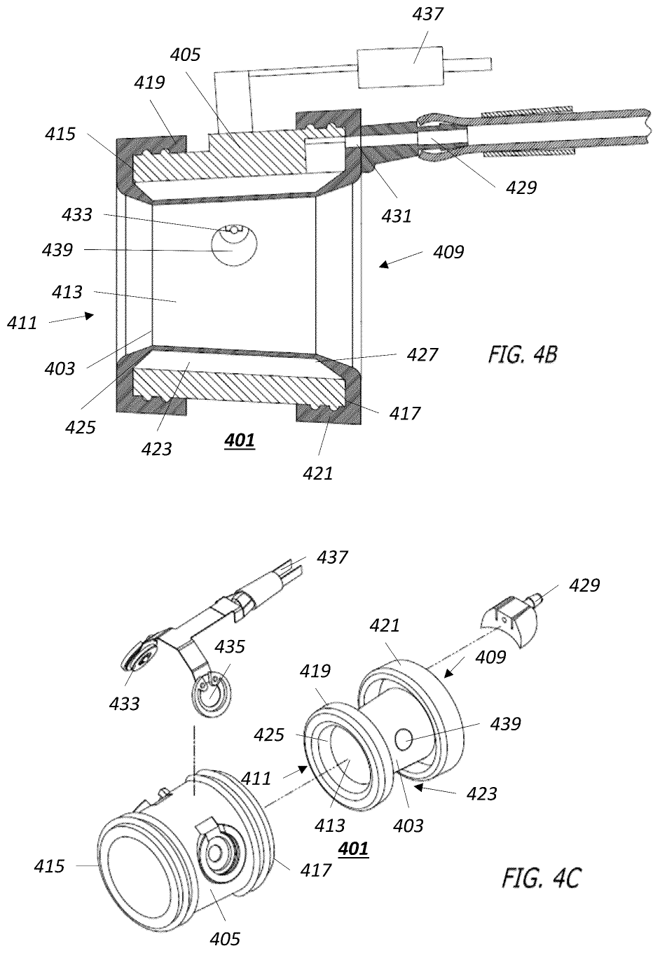

Provided in FIGS. 4A to 4C is a blood pressure cuff 401 that incorporates an enveloped inflatable bladder 403 secured to housing 405 by enveloping over the housing edges. Blood pressure cuff 401 has a tubular shape and is generally constructed to tightly fit onto an extremity. As shown, blood pressure cuff 401 is designed to fit a thumb and includes a knuckle contour 407 that assists in alignment of the sensor systems over the palmar digital arteries to be utilized for hemodynamic measurement. A first tubular end 409 can have a larger diameter than the opposite tubular end 411 to accommodate the contours of the thumb, which can be adapted to fit any extremity. Further, to accommodate a variety of extremity sizes, blood pressure cuff 401 can be generated in variety sizes. For instance, blood pressure cuff 401 can be constructed having a small size, a medium size, and a large size, where the size is correlated with the diameter of the tubular shape.

Inflatable bladder 403 has a tubular shape with a flexible contact sheet 413 that provides contact to the thumb. Inflatable bladder 403 has a folded-over ends 419 and 421 that are folded over a first edge 415 and second edge 417, which is allows for securing to housing 405. Folded-over ends 419 and 421 can be further secured to the housing via a glue or fastener, such as (for example) pins, rivets, or hook. Folded-over ends 419 and 421 provide a sealed inflation chamber 423 in between inflatable bladder 403 and housing 405. It should be understood, however, that any connection for providing a sealed inflation chamber can be utilized.

In between edge 415 and contact sheet 413 is angled segment 425 and in between edge 417 and contact sheet 413 is angled segment 427, forming vertices between the face of the inner contact sheet and the face of each angled segment. Angled segments 425 and 427 each have an angle, a thickness, and density to provide a radially inward push force normal to flexible contact sheet 413 such that blood pressure cuff is secure when fitted on the thumb. Greater thickness of the angled segments also eliminates pressure fluctuations while measuring blood pressure to ensure stable measurement. The push forces also ensure flexible contact sheet 413 has sufficient contact with the thumb when fitted therein. The angle of each vertex between the face of contact sheet 413 and the face of angled segments 425 and 427 can vary, but can generally be between 90 degrees and 150 degrees. The closer the angle is to 90 degrees, the more push force the angled segment can provide. In various implementations, the angle of the vertex between contact sheet 413 and angled segment 425 or between angled segment 427 is between about 80 degrees and about 100 degrees, between about 90 degrees and about 110 degrees, between about 100 degrees and about 120 degrees, between about 110 degrees and about 130 degrees, between about 120 degrees and about 140 degrees, between about 130 degrees and about 150 degrees, or between about 140 degrees and about 160 degrees.

Flexible contact sheet 413 has a thickness that allows elasticity and flexibility such that the inner contact sheet contours to the dimensions of the thumb when inserted within and further to allow expansion and contraction in accordance with inflation and deflation of inflation chamber 423. The thickness of flexible contact sheet 413, angled segment 425, and angled segment 427 depend on the inflatable bladder material and its elasticity, density and/or stiffness. In various instances, the thickness of flexible contact sheet 413 is between 25 μm and 100 μm. In various instances, the thickness of contact sheet 413 is between about 20 μm and about 40 μm, between about 30 μm and about 50 μm, between about 40 μm and about 60 μm, between about 50 μm and about 70 μm, between about 60 μm and about 80 μm, between about 70 μm and about 90 μm, or between about 80 μm and about 100 μm. Angled segment 425 and angled segment 427 can have a tapered thickness such that the thickness adjacent to edges 415 and 417 is greater than the thickness adjacent to the inner contact sheet.

Inflation chamber 423 is in fluidic connection with port 429, which is in connection to the pressure supply pump and pressure regulator system. Folded over end 421 can incorporate an aperture 431 to allow pressure to flow from port 429 into inflation chamber 423. Although port 429 is shown to be located in proximity to tubular end 409, it can be positioned anywhere along the housing as long as it provides a fluidic connection to inflatable chamber 423. Accordingly, in various instances, port 429 is proximal to tubular end 409, port 429 is proximal to tubular end 411, or port 429 is located more centrally between tubular end 409 and tubular end 411.

Blood pressure cuff 401 further incorporates a sensor system comprising a light emitter 433 and a light sensor 435 for sensing arterial diameter or performing other tasks that would be useful in the provision of hemodynamic monitoring (e.g., measuring blood oxygen levels). Light emitter 433 and light sensor 435 are in connection with an electrical connector 437 such that power and data can be transmitted to and from the light emitter and light sensor. As shown, light emitter 433 and light sensor 435 are secured within housing 405, however, the light emitter and light sensor can be provided on the surface of contact sheet 413. When light emitter 433 and light sensor 435 are secured within housing 405, inflatable bladder 403 is to be translucent to the emitted light. Inflatable bladder 403 can incorporate a translucent window 439 or be translucent throughout. Light emitter 433 can be any light source for providing the requisite light wavelength utilized for measuring the arterial diameter, including (but not limited to) a light emitting diode or laser. In some instances, the light emitter can include filter (e.g., narrow band filter) for filtering the provided light wavelength. Likewise, light sensor 435 can be any sensor for detecting light signals, including (but not limited to) a photodiode. In some instances, the system of light emitter 433 and light sensor 435 is a photoplethysmograph.

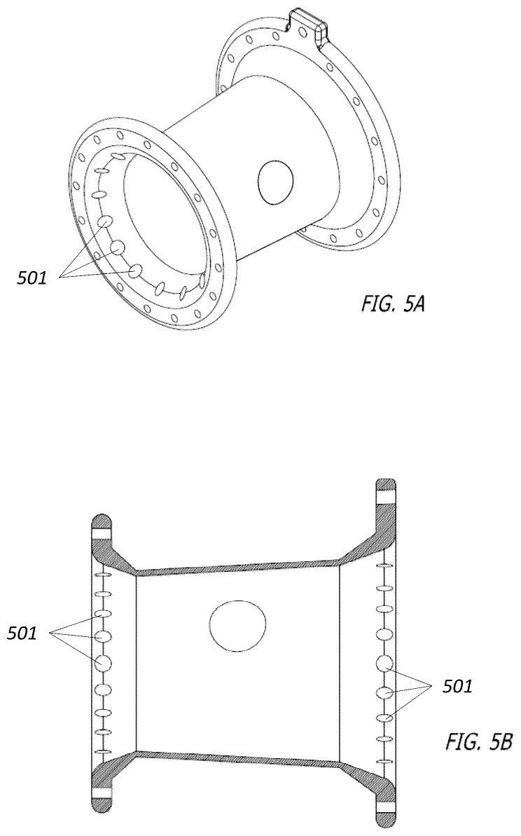

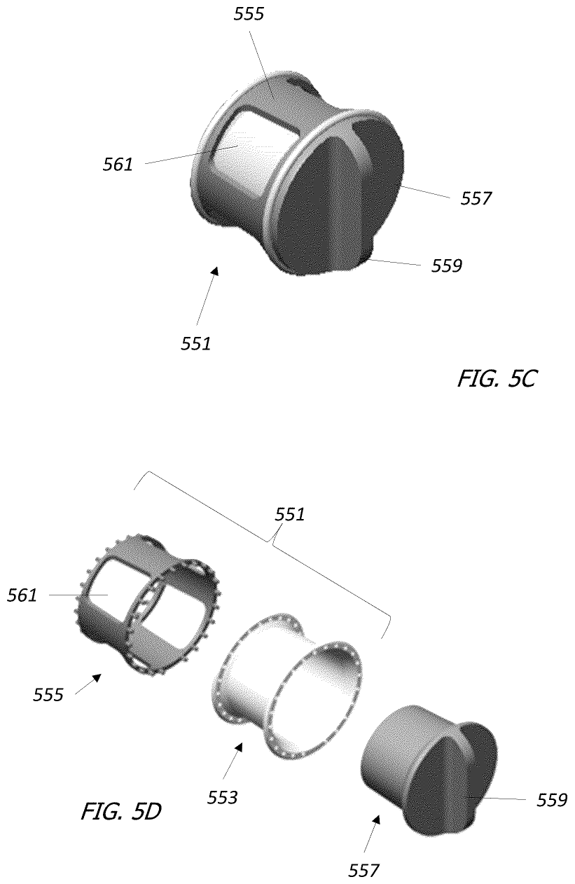

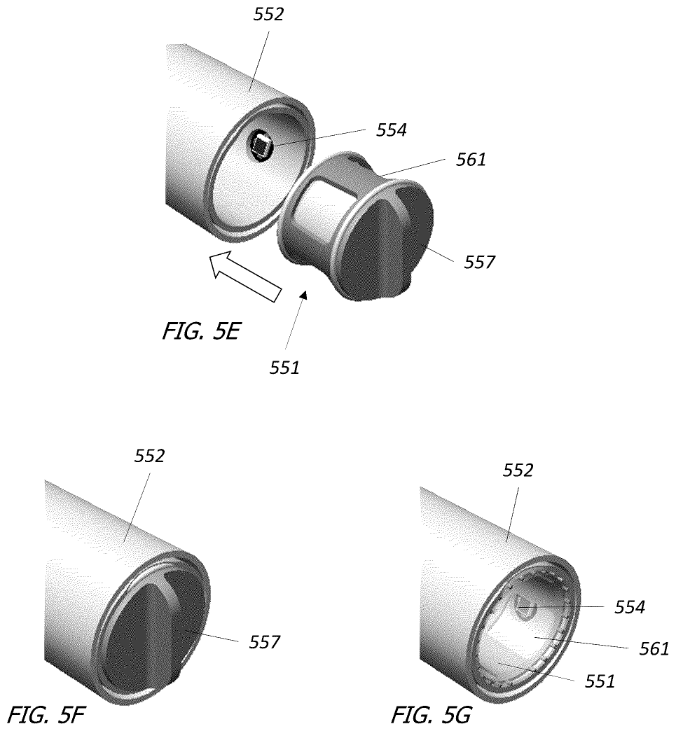

In some implementations, the angled segments of a blood pressure can incorporate one or more features that reduce the push force as the extremity is positioned therein, which can ease the ability cuff to be slid onto a digit or an extremity. In some implementations, one or more furrows are incorporated into the angled segment. Provided in FIGS. 5A and 5B is an inflatable bladder incorporating a plurality of furrows 501 along each of the vertices (also can be referred to as scalloped vertex design). In some instances, only one of the vertices contains one or more furrows. In some instances, the vertex adjacent to the tubular end with a larger aperture contains one or more furrows. In some instances, both vertices each contain one or more furrows. It should also be understood that any alternative means for reducing the push force for the purpose of positioning an extremity within the blood pressure cuff can be utilized.

Provided in FIGS. 5C and 5D is an example of an inflatable bladder system 551 that is cartridge-like and is configured to be removably installed within a housing. Accordingly, inflatable bladder system 551 can be for limited use (e.g., one-time use; or multi-time use) and replaceable with another inflatable bladder system such that the housing can be reutilized after use and removal of each inflatable bladder. Inflatable bladder system 551 comprises an inflatable bladder 553 secured to an outer frame 555, forming an inflation chamber therebetween. Inflatable bladder system 551 can further optionally include a means for grasping and inserting the system into the housing, such as insert 557. Insert 557 is configured to insert within inflatable bladder 553 and includes a grasping portion 559 for facilitating grasping of the system and installing into a housing. In some implementations, the housing includes a light emitter and light sensor for sensing arterial diameter or performing other tasks that would be useful in the provision of hemodynamic monitoring (e.g., measuring blood oxygen levels). When the housing includes a light emitter and light sensor, outer frame 555 and inflatable bladder 553 can incorporate a translucent window 561 or be translucent throughout. Alternatively, a light emitter and light sensor can be provided on outer frame 555 or on inflatable bladder 553. Furthermore, inflatable bladder system 551 can incorporate other features as described for other embodiments, including (but not limited to) a flexible contact sheet, angled segments for providing radially inward push forces, fluidic connection, and electrical connector (see FIGS. 2A to 4C and accompanying description).

Provided in FIGS. 5E to 5G is an example of installing inflatable bladder system 551 within a housing 552. In FIG. 5E, inflatable bladder system 551 is assembled and ready to be installed within housing 552. To install, inflatable bladder system 551 is inserted into housing 552 and aligned such that a light emitter and a light sensor 554 are aligned with translucent window 561. In FIG. 5F, inflatable bladder system 551 is installed within housing 552. Housing 552 and/or inflatable bladder system 551 can include a means for alignment and/or securement (e.g., clips, guide posts, asymmetrical fittings, etc.). In FIG. 5G, optional insert 557 is removed, readying the inflatable bladder system 551 to perform hemodynamic monitoring. Upon completion of use of inflatable bladder system 551, it can be removed and replaced with another inflatable bladder system.

Various implementations of blood pressure cuffs incorporate one or more stabilizers. When the cuff is placed onto a digit or extremity, minor movements such as slight twisting or sliding movements can alter the light emitter and a light sensor accuracy, resulting in faulty blood pressure readings. To better ensure immobility to prevent minor movements, the cuff can incorporate one or more stabilizers within the inflation chamber. A stabilizer can secure the digit by providing a slightly stronger push force against the digit or extremity. The one or more stabilizers should be configured such that they do not interfere with the ability of the bladder to inflate, interfere with the passage of the light signals, or alter the blood flow within the digit or extremity. Accordingly, stabilizers should allow pressure to flow freely within the bladder. Stabilizers should also be positioned to not block the light path or include a window to allow the light traverse. And stabilizers should also provide enough push forces to prevent movement of the cuff while on the digit or extremity but should not have so much push force that it disrupts blood flow or changes the diameter of the artery.

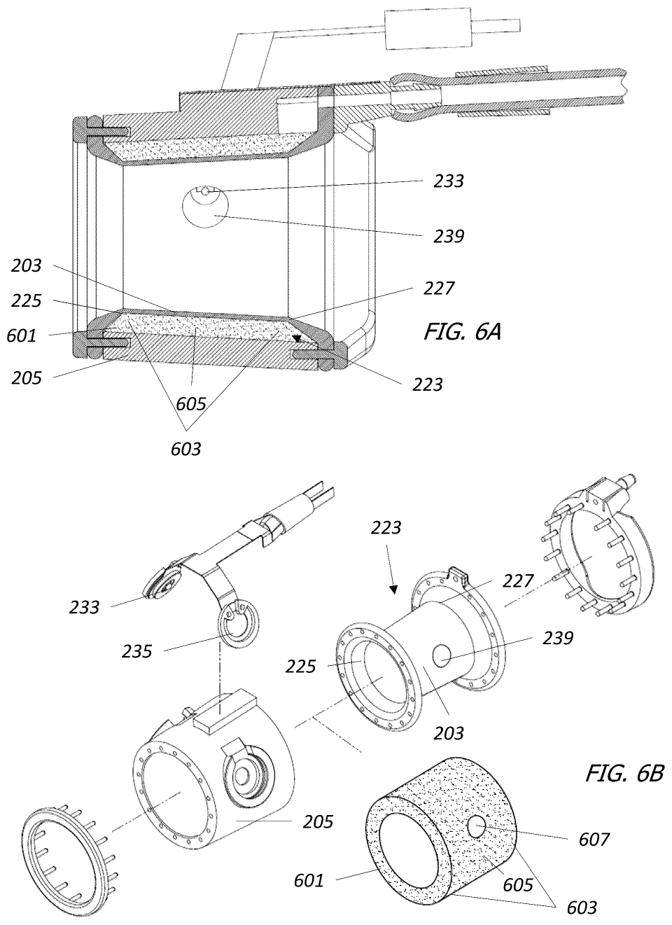

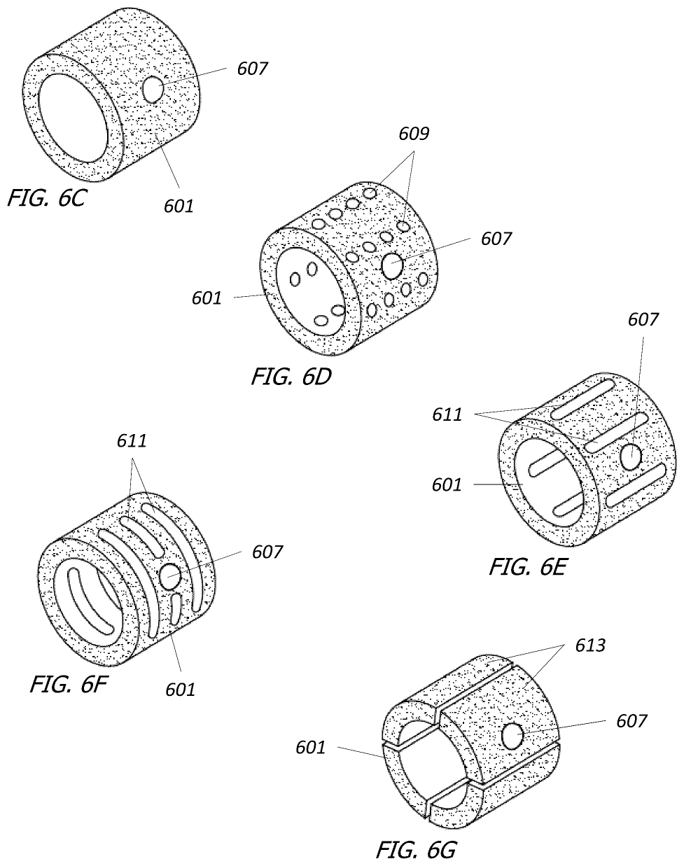

Various designs and configurations of stabilizers can be utilized, such as (for example) a sponge or foam fill, a set of springs, or a set of columnar supports. FIGS. 6A to 9B provide examples of various stabilizers that can be utilized in various implementations of the blood pressure cuff configurations as described herein, such as the various configurations depicted in FIGS. 2A to 5D. It should be further understood that stabilizers shown and described are merely examples and various other designs and configurations of stabilizers can be utilized, as can be inferred from the drawings and descriptions provided. Further, any of the described stabilizers can be configured to be combined with any other stabilizer described herein or otherwise. For instance, the sponge or foam fill can include spacing within the fill for a set of springs and/or a set of columnar supports. Likewise, a set of springs can be positioned to allow space for a set of columnar supports and vice versa.

FIGS. 6A to 6G provide examples of sponge or foam fill stabilizers that can fill the space within the inflation chamber. As can be seen in FIGS. 6A and 6B, the sponge or foam fill 601 is within inflation chamber 223, between housing 205 and bladder 203. The sponge or foam fill can fill a portion or all of the inflation chamber, but should have a density such that it can provided push forces onto a digit to stabilize the digit within the cuff aperture but also allow air pressure to fill within the chamber for blood pressure measurement. In some implementations, the density of the foam can be varied such that it is denser and provide stronger push forces in some areas. In some implementations, a gradient of density is provided such that the foam is most dense in the region 603 adjacent to the angled segments 225 and 227 and is less dense within a central region 605 of inflatable bladder, which may have the benefit of solidifying grasp on the digit or extremity while allowing for proper air pressure flow at the central portion of the cuff where pressure is put onto the digit or extremity to perform the blood pressure measurements.

Sponge or foam fill 601 can have a variety designs and implementations. Generally, a sponge or foam fill will have a tubular shape and fit within and between the housing and inflation chamber of the digit cuff. As shown in FIGS. 6A and 6B, when the cuff implements light emitter 233 and light sensor 235 in the housing, sponge or foam fill can include one or more windows 607 or other means to allow the light to transfer through inflation chamber 223 to perform signal measurements upon the arteries of the digit or extremity. Window 607 can be an open aperture or composed of a material translucent to the signal wavelength. Window 607 should be aligned with light emitter 233 and light sensor 235 and with translucent window 239 of the inflatable bladder. Alternatively, the light emitter and the sensor can be provided on the bladder (see FIGS. 2D to 2F) and the sponge or foam fill will need to allow the electrical connectors of the light emitter and the sensor to transfer therethrough.

The designs and implementations of sponge or foam fill 601 can include a one or more apertures 609, one or more slits 611, and/or one or more segments 613. Various design or implementations can vary the air pressure flow, rigidity, viscoelasticity, and push forces provided by the fill or foam. The one or more apertures 609 and the one or more slits 611 can ease the ability of air pressure to flow within inflatable chamber 223, which can enhance the ability to of the chamber to inflate and deflate at an appropriate rate to perform hemodynamic measurements.

Provided in FIGS. 6C to 6G are varied implementations of sponge or foam fill 601. The sponge or foam fill can be substantially filled with minimal number of apertures and slits, but can still include one or more windows and/or apertures to allow light transmission therethrough via the light emitter and light sensor (FIG. 6C). In some implementations, sponge or foam fill 601 includes one or more apertures 609, which can be provided as a patterned array, a randomized array, or any other configuration (FIG. 6D). In some implementations, sponge or foam fill 601 includes one or more slits 611 which can be provided as a patterned array, a randomized array, or any other configuration (FIGS. 6E and 6F). In some implementations, slit 611 is provided along the tubular axis (i.e., the elongated axis the cuff) (FIG. 6E) or in an axis perpendicular to the tubular axis (FIG. 6F), or in any other axis. In some implementations, sponge or foam fill 601 is provided as multiple segments 613 (FIG. 6G). Any number of segments having any shape and/or any interconnection among the segments can be utilized. Although specific designs and implementations are shown in FIGS. 6C to 6G, it is to be understood that any design or implementation can be utilized. Further, a design or implementation can include any combination of apertures, slits, and/or segments. Designs and implementations can also include one or more ridges and/or one or more to furrows in addition to or as a replacement of the apertures, slits, and segments as depicted and described.

Sponge or foam fill can be comprised of any material capable of providing firmness and push forces while allowing air pressure flow therethrough. The sponge or foam should exhibit robust viscoelasticity such that it can conform to the shape of an object when compressed and fitted and further capable of resuming its initial shape upon release. Specifically, the sponge or foam should be able to fit and conform to a digit or other extremity when in use but also be able to reform to its initial shape after use.

The sponge or foam fill should also have ability to allow air pressure flow yet still have robust viscoelasticity. In many implementations, the fill is an open cell sponge or foam, wherein at least 50% of the cells of the sponge or foam are open. In various implementations, the fill is an open cell sponge or foam having between about 40% open cells and about 60% open cells, the fill is an open cell sponge or foam having between about 50% open cells and about 70% open cells, the fill is an open cell sponge or foam having between about 60% open cells and about 80% open cells, the fill is an open cell sponge or foam having between about 70% open cells and about 90% open cells, or the fill is an open cell sponge or foam having between about 80% open cells and about 100% open cells. In reference to percentage of open cells, “about” is to mean±5%. Materials for a sponge or foam fill can be any material that can be formed into a sponge or foam with the properties of rigidity, robust push forces, robust viscoelasticity, and capable of allowing air pressure to flow within. Examples of materials that can be utilized to yield a viscoelastic sponge or foam include (but are not limited to) polytetrafluoroethylene (PTFE), polypropylene (PP), ethylene tetrafluoroethylene (ETFE), polyethylene (PE), high density polyethylene (HDPE), polyethylene terephthalate (PET), polyvinylchloride (PVC), polystyrene (PS), polyurethane (PU), ethylene propylene diene monomer (EPDM), isobutylene isoprene, neoprene, rubber, nitrile, latex, and polysiloxane. Further, as is well appreciated in the art, the viscoelasticity and firmness of various material and polymers can be altered by chemical modification, incorporating reinforced components, blending various materials, or altering density.

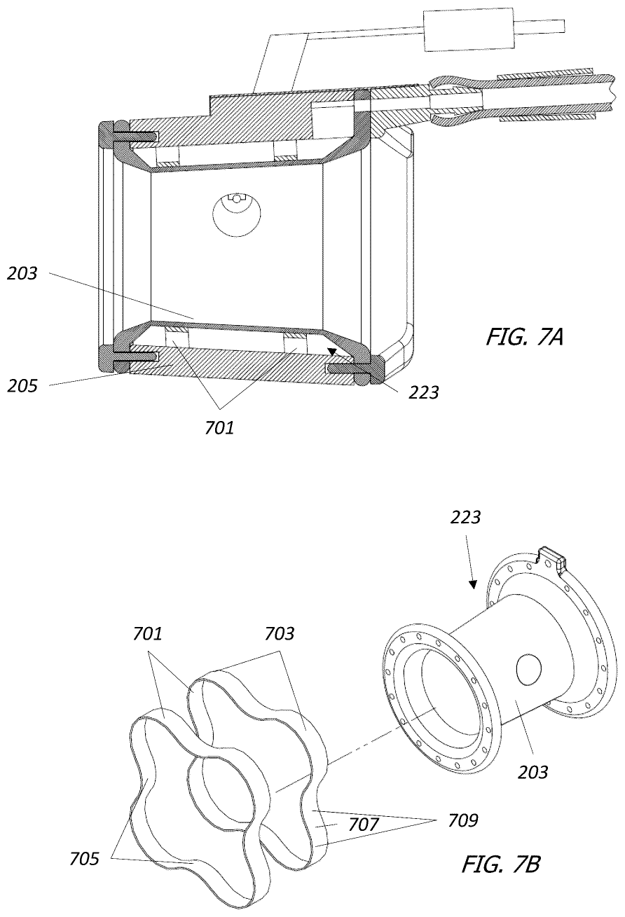

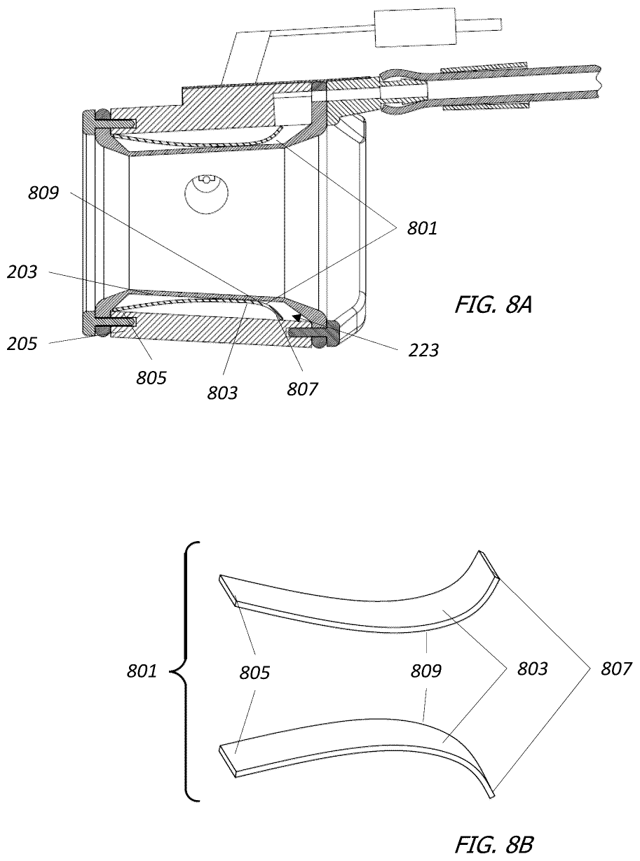

In some implementations, a set of one or more springs having linear elasticity is utilized as a stabilizer. FIGS. 7A to 8B provide two examples of resilient springs that can be utilized to stabilize a digit or an extremity with a cuff. In some implementations, the spring is a clover-shaped ring that encircles the inflatable bladder of the cuff (FIGS. 7A and 7B). In some implementations the spring is an extended and curved ribbon that extends along the tubular axis (i.e., elongated axis of the cuff) (FIGS. 8A and 8B).

Provided in FIGS. 7A and 7B are examples of blood pressure cuffs having a set of two clover-shaped rings 701 to provide additional support for securing the cuff on the digit or extremity. Each clover-shaped ring 701 fits within inflation chamber 223, between inflatable bladder 203 and housing 205. Each clover-shaped ring 701 can be an undulating ribbon that has multiple contacts with housing 205 such that an outer face of each clover-shaped ring contacts the inner wall of the housing at multiple points to form multiple contacts 703 with the housing. Each clover-shaped ring 701 can further include inner face having multiple points that are in contact with or adjacent to the surface of inflatable bladder 203 at multiple points to form multiple contacts or adjacencies 705 with the housing. In many implementations, the contacts 703 between each clover-shaped ring 701 and housing 205 are secured such that the ring is fixed in its place, which can be done by utilizing an adhesive, an interlocking joint, or any other mechanism for securement. Any contacts between each clover-shaped ring 701 and inflatable bladder 203 may or may not be secured, which may depend on the ability to secure a digit or extremity therein but still allow the bladder to inflate freely for blood pressure measurement.

Each clover-shaped ring 701 can have some spring resiliency, such that a digit or extremity is able to be slid within and initially fitted but capable of providing push forces to prevent small movements once the digit or extremity is fitted therein. To provide resiliency, an interconnecting portion 707 can flex when pushed upon and then spring back once the push force is released. Each interconnecting portion 707 includes a set of two hinges 709, each hinge adjacent to either the contact between contacts 703 and housing 205 and the contact or the adjacency 705 between inner face and inflatable bladder 203. Each hinge of the set of hinges 709 can have an angle between about 30 degrees and about 150 degrees. The degree angle of the hinge can influence the amount of push force each clover-shaped ring 701 provides.

The number of clover-shaped rings and the width and the thickness of each clover-shaped ring can be dependent on the amount of push force needed and the level of resiliency to be provided. Greater number, greater width, and greater thickness will provide stronger push forces. Ultimately, the amount of push force to be provided will be a balance between the ability to secure a digit or extremity therein but not disrupt the ability of the bladder to inflate freely and control the dynamics of the artery within the digit or extremity for blood pressure measurement.

The clover-shaped rings should be composed of material with high resiliency or high linear elasticity. In many implementations, a highly resilient metal is utilized. Highly resilient metals include (but are not limited to) steel, nickel, copper, titanium, aluminum, and alloys thereof. In some implementations, linear-elastic nitinol is utilized, which can better resist deformation than shape-memory nitinol.

Provided in FIGS. 8A and 8B are examples of blood pressure cuffs having set of two elongated ribbons 801 to provide additional support for securing the cuff on the digit or extremity. Each elongated ribbon 801 fits within inflation chamber 223, between inflatable bladder 203 and housing 205, and is elongated in the direction of the tubular axis. Each elongated ribbon 801 can include a curved portion 803 and a secured end 805 that is secured to housing 205. Opposite of secured end 805 is an unsecured end 807 that is free to move in a direction along the tubular axis. In many implementations, secured end 805 of each elongated ribbon 801 is secured housing 205 such that the ribbon is fixed in its place, which can be done by utilizing an adhesive, an interlocking joint, or any other mechanism for securement. Elongated ribbon can further include a contact or an adjacency at the apex 809 of curved portion 803 that is contact with or adjacent to inflatable bladder 203. Any contact point between each elongated ribbon 801 and inflatable bladder 203 may or may not be secured, which may depend on the compromise between the ability to secure a digit or extremity therein but still allow the bladder to inflate freely for blood pressure measurement.

Each elongated ribbon 801 can have some spring resiliency, such that a digit or extremity is able to be slid within and initially fitted but capable of providing push forces to prevent small movements once the digit or extremity is fitted therein. To provide resiliency, curved portion 803 between can flex when pushed upon and then spring back once the push force is released. When curved portion 803 flexes, unsecured end 807 can move along the tubular axis to allow deformation of the elongated ribbon.

The number of elongated ribbons and the width and the thickness of each elongated ribbon can be dependent on the amount of push force needed and the level of resiliency to be provided. Greater number, greater width, and greater thickness will provide stronger push forces. Ultimately, the amount of push force to be provided will be a balance between the ability to secure a digit or extremity therein but not disrupt the ability of the bladder to inflate freely and control the dynamics of the artery within the digit or extremity for blood pressure measurement. Further, the number of elongated ribbons and the spacing between the elongated ribbons may be provided such that the combined push force that is generated yields a radially inward force.

The elongated ribbon should be composed of material with high resiliency or high linear elasticity. In many implementations, a highly resilient metal is utilized. Highly resilient metals include (but are not limited to) steel, nickel, copper, titanium, aluminum, and alloys thereof. In some implementations, linear-elastic nitinol is utilized, which can better resist deformation than shape-memory nitinol.

In some implementations, a set of one or more columnar supports (or a radial support) is utilized as a stabilizer. Each columnar support can be elastomeric and can strengthen the push forces provided by the angled segments of the inflatable bladder by providing a columnar extension from the housing to vertex between the angled segment and contact sheet. The columnar extension can be molded along with the inflatable bladder or constructed independently and installed.

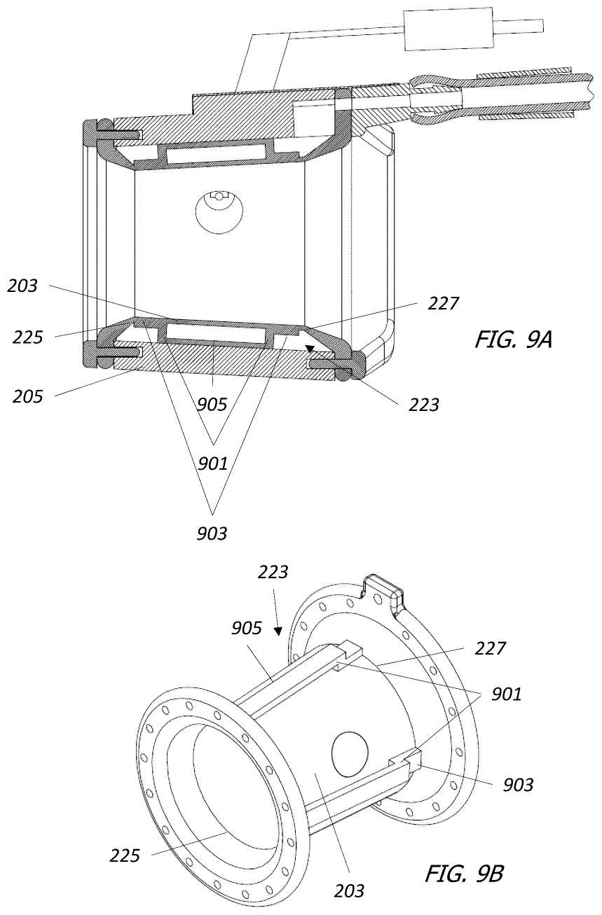

Provided in FIGS. 9A and 9B is an example of an inflatable bladder 203 having a set of columnar supports 901 that strengthen the push forces of angled segments 225 and 227. Each columnar support 901 includes a contact point 903 with a vertex adjacent to angled segment 225 or 227 and extends to and is secured to the inner face of housing 205. Each columnar support 901 can include a base 905 to provide a point of contact to secure the columnar support to the housing. Base 905 can be secured to housing 205 via an adhesive, an interlocking joint, a set of one or more rivets, or any other mechanism for securement. Although base 905 is shown to extend along the housing, the base can be provided in any shape that can provide securement to the housing and able to support columnar support 901. Further, although columnar support 901 is shown to be perpendicular from the housing wall to the inner contact sheet, the columnar support can be provided at an angle from about 45-degrees to about 90-degrees. The angle of the columnar support can impact the amount of the push force that is provided.

The number of columnar supports and the width and the thickness of each columnar support can be dependent on the amount of push force needed and the level of resiliency to be provided. Greater number, greater width, and greater thickness will provide stronger push forces. And in some implementations, a radial support extending along the inner circumference of the housing is utilized. Ultimately, the amount of push force to be provided will be a balance between the ability to secure a digit or extremity therein but not disrupt the ability of the bladder to inflate freely and control the dynamics of the artery within the digit or extremity for blood pressure measurement. The number of columnar supports and the spacing between the columnar supports may be provided such that the combined push force that is generated yields a force vector towards the center of the cuff.

The set of columnar supports can be composed of an elastomeric material. Several materials and/or polymers are known to be elastomeric and can be used as a columnar support (or radial support), including (but not limited to) polytetrafluoroethylene (PTFE), polypropylene (PP), ethylene tetrafluoroethylene (ETFE), polyethylene (PE), high density polyethylene (HDPE), polyethylene terephthalate (PET), polyvinylchloride (PVC), polystyrene (PS), polyurethane (PU), ethylene propylene diene monomer (EPDM), isobutylene isoprene, neoprene, rubber, nitrile, latex, and polysiloxane. In some implementations, the inflatable bladder and the set of columnar supports are composed of the same material, especially when constructed as a single mold.

### Light Emission and Sensing

To measure the diameter of the artery for blood pressure measurement, a light emitter and a light sensor is utilized. In many instances, a photoplethysmograph system is utilized. The light emitter and light sensor can generally be positioned anywhere within the blood pressure cuff as long as the light emitter is capable of emitting light towards the artery and the light sensor is capable of receiving light reflected off the artery. In many instances, the light emitter and the light sensor are centrally located between the tubular edges and laterally aligned.

A blood pressure cuff can also incorporate a plurality of light emitters and/or a plurality of light sensors. There are various benefits to having multiple light emitters and/or light sensors. For instance, having two or more light emitters can allow for two or more wavelengths of light to be emitted, each emitter having a unique emission wavelength. While standard blood pressure measurement typically only uses one wavelength of light to measure arterial diameter, other measurements can require two or more wavelengths. For example, methods to measure oxygen saturation levels typically utilizes two different wavelengths.

In some instances, it can be beneficial to have multiple light sensors. For example, it can be beneficial to have multiple light sensors to improve the permissibility of various extremity sizes. In other words, having two light sensors can allow the cuff to measure larger extremities with a first sensor and smaller extremities with a second sensor. Generally, for increasing the permissibility of extremity sizes, the two or more sensors would be adjacent or near one another with the light emitter on the opposite side of the cuff. Having multiple light sensors can also provide a means for ensuring that the cuff is positioned correctly.

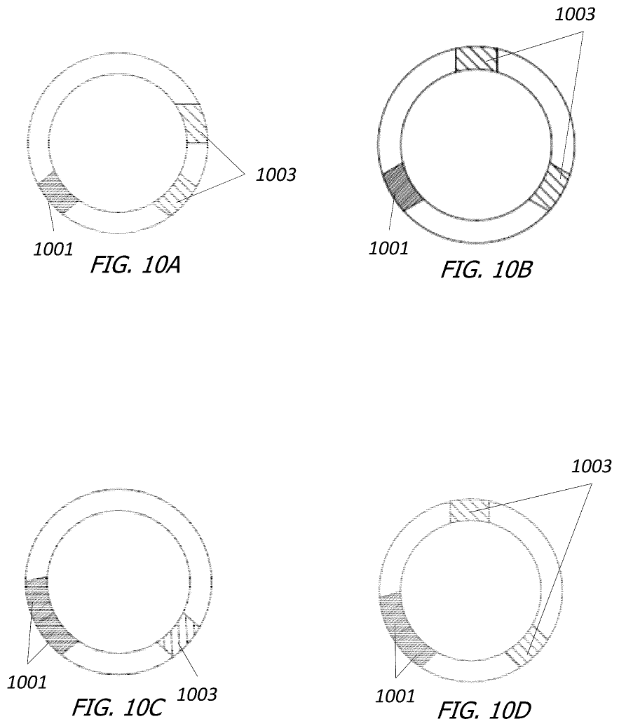

Provided in FIGS. 10A to 10D are various depictions of a blood pressure cuff having a plurality of light emitter and/or a plurality of light sensors in various orientations. Generally, the light emitters and light sensors are laterally aligned but can vary in their position around the cuff within the lateral plane.

FIG. 10A depicts a blood pressure cuff having a single light emitter 1001 and two light sensors 1003. The light sensors are adjacent (or within proximity) to one another at around the 3:00 and 4:30 positions with the light emitter at around the 7:30 position. This positioning can allow for greater permissibility of various extremity sizes that the cuff can measure. It should be understood that the light sensors and light emitter can moved to any position around the clock while maintaining the same approximate spacing among the sensors and emitters.

FIG. 10B depicts a blood pressure cuff having a single light emitter 1001 and two light sensors 1003. The light sensors are kept distant from one another at around the 4:30 and 12:00 positions with the light emitter at around the 7:30 position. This positioning can allow for determining whether the cuff has been fitted onto the extremity correctly. If the sensor at 12:00 is receiving light signals, it suggests that the cuff is not fitted correctly. It should be understood that the light sensors and light emitter can moved to any position around the clock while maintaining the same approximate spacing among the sensors and emitters.

FIG. 10C depicts a blood pressure cuff having two light emitters 1001 and a single light sensor 1003. The light emitters are adjacent (or within proximity) to one another at around the 7:00 and 8:00 positions with the light sensor at around the 4:30 position. Each light emitter can emit its own unique wavelength of light. This positioning can allow for each light emission to utilize the same sensor for detection. Accordingly, this positioning can be utilized for performing both blood pressure and oxygen saturation measurements. It should be understood that the light emitters and light sensor can moved to any position around the clock while maintaining the same approximate spacing among the emitters and sensors.

FIG. 10D depicts a blood pressure cuff having two light emitters 1001 and two light sensors 1003. The light emitters are adjacent (or within proximity) to one another at around the 7:00 and 8:00 positions with the light sensors at around the 4:30 position and 12:00 position. Each light emitter can emit its own unique wavelength of light. This positioning can be utilized for performing both blood pressure and oxygen saturation measurements and also allow for determining whether the cuff has been fitted onto the extremity correctly. It should be understood that the light emitters and light sensor can moved to any position around the clock while maintaining the same approximate spacing among the emitters and sensors.

### Methods of Use

The various blood pressure cuffs of the disclosure can be utilized to measure blood pressure. In many instances, the blood pressure cuffs are utilized for continuous blood pressure monitoring. Generally, to perform continuous blood pressure monitoring, the blood pressure cuff is fitted and positioned onto the individual's extremity. Once positioned, a computational program can be utilized to continuously measure arterial blood pressure within the extremity and further calculate a more central arterial blood pressure.

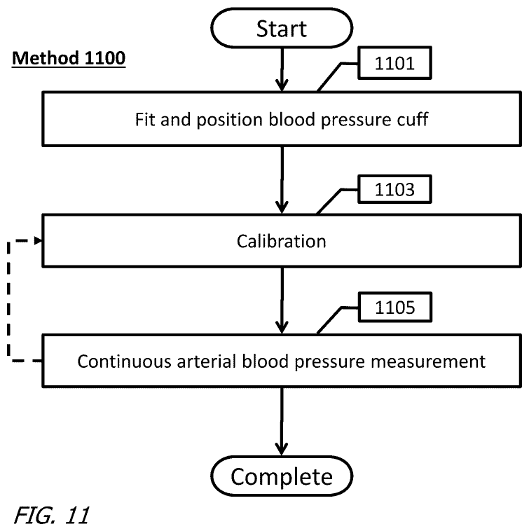

Provided in FIG. 11 is an example of a method of utilizing a blood pressure cuff. Method 1100 can begin by fitting and positioning (1101) the blood pressure cuff onto an extremity, such as a finger or thumb. To fit the blood pressure cuff, the cuff is slid onto the extremity and down to the position to perform measurement. In some instances, the blood pressure cuff has a tubular shape with apertures contoured to the extremity. For example, a blood pressure cuff for fitting on the thumb may have ovular apertures that match the general contours of a thumb, including contours of knuckles. Accordingly, fitting of the blood pressure cuff onto the thumb may require that the cuff is positioned such that the contours of the cuff are aligned and fitted with the contours of the extremity of which the cuff is to be fitted upon.

Upon fitting the blood pressure cuff to the extremity, blood pressure measurement may commence. Method 1100 performs (1103) an initial calibration to determine a mean arterial pressure to yield an unloaded volume (i.e., the volume to yield a constant arterial diameter). After initial calibration, method 1100 continuously measures (1105) arterial blood pressure of the extremity. Further intermittent calibration can be performed (1103) to further update the calculation of a mean arterial pressure to yield unloaded volume such that continuous measurement of arterial blood pressure remains accurate. The initial and intermittent calibrations and continuous measurements can be performed by a computational processing system.

### Computational Processing System

A computational processing system for blood pressure monitoring via a blood pressure cuff can utilize a processing system including one or more of a CPU, GPU and/or neural processing engine. A computational processing system can be utilized to measure arterial blood pressure by sensing an arterial diameter via a sensor system and regulating pressure within the blood pressure cuff to yield an unloaded volume.

The computational processing system can be housed within a patient monitor in a direct wired connection between the monitor and components, inclusive of a pressurized digit cuff, photoplethysmograph, a pressure pump system, and a pressure regulator. Alternatively, the computational processing system can be housed separately from the patient monitor and components, utilizing a wireless connection (e.g., WiFi, cellular, Bluetooth, etc). The computational processing system can be implemented on any appropriate computing device, including (but not limited to) a dedicated computer, portable computer, and/or tablet.

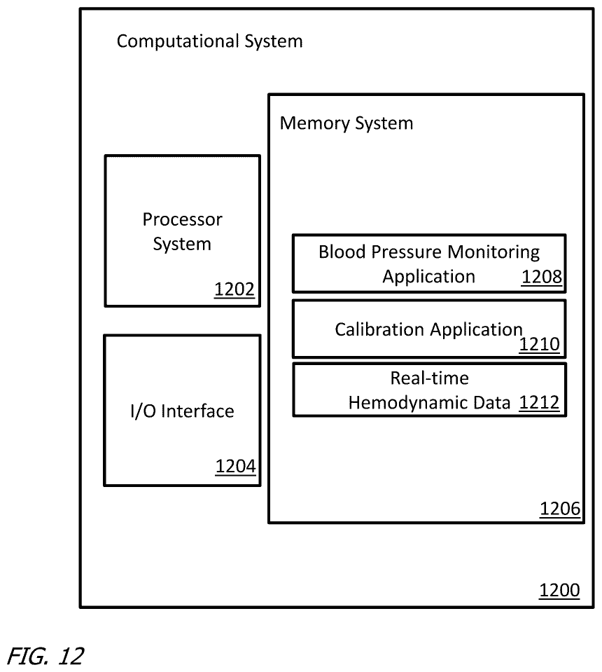

An exemplary computational processing system that can be utilized to perform the various methods and processes of the disclosure is illustrated in FIG. 12. The computational processing system 1200 includes a processor system 1202, an I/O interface 1204, and a memory system 1206. As can readily be appreciated, the processor system 1202, I/O interface 1204, and memory system 1206 can be implemented using any of a variety of components appropriate to the requirements of specific applications including (but not limited to) CPUs, GPUs, ISPs, DSPs, wireless modems (e.g., WiFi, Bluetooth modems), serial interfaces, volatile memory (e.g., DRAM) and/or non-volatile memory (e.g., SRAM, and/or NAND Flash).

In the illustrated example, the memory system is capable of storing various computational applications for instructing a blood pressure monitoring system to perform blood pressure measurements and calibrations. It is to be understood that the blood pressure monitoring and calibration applications are a representative sample of what can be stored in memory.

In some implementations, the memory system 1206 can store a blood pressure monitoring application 1208 and a calibration application 1210, which can be utilized to perform processes to perform blood pressure measurements and calibrations. Generally, the blood pressure monitoring application can interpret light signals derived from the sensor system and direct the pressure regulator system to provide a requisite pressure amount to yield an unloaded volume. Further, a calibration application can be utilized to direct the pressure regulator system to run a calibration process that adjusts the provided pressure in order to determine a mean arterial pressure representative of the unloaded volume. In some implementations, the memory system can further store real-time hemodynamic data 1212 that can be displayed on a dispay screen via the I/O interface 1204.

While specific computational processing systems are described above with reference to FIG. 12, it should be readily appreciated that computational processes and/or other processes utilized in the provision of continuous blood pressure monitoring can be implemented on any of a variety of processing devices including combinations of processing devices. Accordingly, computational devices should be understood as not limited to specific monitoring systems, computational processing systems, and/or specific applications and models. Computational devices can be implemented using any of the combinations of systems described herein and/or modified versions of the systems described herein to perform the processes, combinations of processes, and/or modified versions of the processes described herein.

Example 1. A blood pressure cuff for use in a blood pressure measurement system, comprising:
a housing and an inflatable bladder, the inflatable bladder in connection with the housing such that an inflation chamber is formed in between the inflatable bladder and the housing, wherein the inflatable bladder comprises an elastomeric molded material in a tubular shape, wherein the housing is fixed in a tubular shape;
wherein the inflatable bladder is contoured to conform to an extremity of an individual;
wherein the inflatable bladder comprises an inner contact sheet having a tubular shape, a set of angled segments, and two tubular edges; wherein each angled segment is in between the inner contact sheet and one of the tubular edges of the inflatable bladder.

Example 2. The blood pressure cuff of example 1, wherein each angled segment has an angle, density and a thickness for providing a push force, and wherein the thickness of each angled segment is greater than the thickness of the inner contact sheet.

Example 3. The blood pressure cuff of example 2, wherein a vertex is formed between a face of each angled segment and a face of the inner contact sheet; wherein each vertex independently has an angle between 90 degrees and 150 degrees.

Example 4. The blood pressure cuff of example 2 or 3, wherein each angled segment has a tapered thickness such that the thickness proximal to the tubular edge is greater than the thickness proximal to the inner contact sheet.

Example 5. The blood pressure cuff of any one of examples 1 to 4, wherein the inner contact sheet is composed of flexible and has thickness such that it can expand and contract in accordance with inflation and deflation of the inflation chamber

Example 6. The blood pressure cuff of example 5, wherein the inner contact sheet has a thickness between 25 μm and 100 μm.

Example 7. The blood pressure cuff of any one of examples 1 to 6, wherein the inflatable bladder is a single molded material comprising the inner contact sheet and the set of angled segments.

Example 8. The blood pressure cuff of any one of examples 1 to 7, wherein the inflatable bladder is a welded set of components comprising the inner contact sheet and the set of angled segments.

Example 9. The blood pressure cuff of any one of examples 1 to 7 further comprising a set of two end caps, wherein the inflatable bladder further comprises a flap at each tubular end, wherein the set of end caps is utilized to secure the inflatable bladder to the housing via each flap.

Example 10. The blood pressure cuff of any one of examples 1 to 7, wherein the inflatable bladder comprises a set of two folded-over ends that each fold over an edge of the housing.

Example 11. The blood pressure cuff of any one of examples 1 to 7 further comprising an outer frame secured to the inflatable bladder, wherein the inflation chamber is further formed between the outer frame and the inflatable bladder; wherein the outer frame and inflatable bladder are configured to be removably installed within the housing such that the housing can be reused after use and removal of the inflatable bladder.

Example 12. The blood pressure cuff of any one of examples 1 to 11, wherein the inflatable bladder comprises a set of one or more furrows along at least one vertex between the inner contact sheet and an angled segment of the set of angled segments.

Example 13. The blood pressure cuff of any one of examples 1 to 12, wherein the housing comprises a fluid port that is in fluidic connection with the inflation chamber.

Example 14. The blood pressure cuff of any one of examples 1 to 13 further comprising a stabilizer within the inflation chamber.

Example 15. The blood pressure cuff of example 14, wherein the stabilizer is a sponge or foam fill.

Example 16. The blood pressure cuff of example 15, wherein the sponge or foam fill contains at least 50% open cells.

Example 17. The blood pressure cuff of example 15 or 16, wherein the sponge or foam fill is composed of material to yield a viscoelastic sponge or foam.

Example 18. The blood pressure cuff of example 15, 16, or 17, wherein the sponge or foam fill has a density gradient.

Example 19. The blood pressure cuff of example 18, wherein the sponge or foam fill has greater density near the angled segments of the inflatable bladder and less density near the center of the inflatable bladder.

Example 20. The blood pressure cuff of any one of examples 15 to 19, wherein the sponge or foam fill includes one or more windows to allow light signals to pass therethrough.

Example 21. The blood pressure cuff of any one of examples 15 to 20, wherein the sponge or foam fill includes one or more apertures, one or more slits, or one or more segments.

Example 22. The blood pressure cuff of example 14, wherein the stabilizer is a set of one or more springs.

Example 23. The blood pressure cuff of example 22, wherein each spring of the set is a clover-shaped ring.

Example 24. The blood pressure cuff of example 23, wherein each clover-shaped ring contacts and is secured to the housing at multiple points, wherein each clover-shaped ring contacts or is adjacent to the inflatable bladder at multiple points, wherein each clover-shaped ring includes an interconnecting portion between each contact with the housing and each contact or adjacency with the inflatable bladder, and wherein each interconnecting portion includes a first hinge near the contact with the housing and a second hinge near the contact or adjacency with the inflatable bladder.

Example 25. The blood pressure cuff of example 24, wherein the first hinge and the second hinge of each clover-shaped ring each individually have an angle between about 30 degrees and about 150 degrees.

Example 26. The blood pressure cuff of example 22, wherein each spring of the set is an elongated ribbon that is elongated in a direction along a tubular axis of the blood pressure cuff.

Example 27. The blood pressure cuff of example 26, wherein each elongated ribbon includes a secured end that is secured to the housing, an unsecured end that is free to move in a direction along the tubular axis, and a curved portion between the secured end and the unsecured end.

Example 28. The blood pressure cuff of example 27, wherein an apex of the curved portion is in contact with or adjacent to the inflatable bladder.

Example 29. The blood pressure cuff of any one of examples 22 to 28, wherein each spring of the set is composed a material with high resiliency or high linear elasticity.

Example 30. The blood pressure cuff of example 14, wherein the stabilizer is a set of one or more columnar supports.

Example 31. The blood pressure cuff of example 30, wherein each columnar support of the set of columnar supports is in contact with a vertex adjacent to an angled segment of the set of angled segments and further in contact with and secured to the housing.

Example 32. The blood pressure cuff of example 31, wherein each columnar support of the set of columnar supports comprises a base, wherein each base provides contact is secured to the housing.

Example 33. The blood pressure cuff of example 31 or 32, wherein each columnar support of the set of columnar supports is individually provided at an angle from about 45-degrees to about 90-degrees from the housing to the vertex.

Example 34. The blood pressure cuff of any one of examples 30 to 31, wherein each columnar support is composed of an elastomeric material.

Example 35. The blood pressure cuff of any one of examples 30 to 32, wherein the set of one or more columnar supports and the inflatable bladder are a single mold.

Example 36. The blood pressure cuff of any one of examples 1 to 35 further comprising a sensor system comprising light emitter and a light sensor.

Example 37. The blood pressure cuff of example 36, wherein the sensor system is a photoplethysmograph.

Example 38. The blood pressure cuff of example 36 or 37, wherein the light emitter is light emitting diode or a laser.

Example 39. The blood pressure cuff of example 36, 37, or 38, wherein the light sensor is a photodiode.

Example 40. The blood pressure cuff of any one of examples 36 to 39, wherein the sensor system comprises at least light sensors positioned such that the sensor system can be utilized to measure blood pressure of various sizes of extremities.

Example 41. The blood pressure cuff of any one of examples 36 to 40, wherein the sensor system comprises at least two light sensors positioned such that the sensor system can be utilized to determine whether the blood pressure cuff has been fitted correctly.

Example 42. The blood pressure cuff of any one of examples 36 to 41, wherein the sensor system comprises at least two light emitters, each light emitter emitting a unique wavelength of light.

Example 43. The blood pressure cuff of example 42, wherein the at least two light emitters are position such that the sensor system can be utilized to measure blood oxygen saturation in addition to measuring blood pressure.

Example 44. The blood pressure cuff of any one of examples 36 to 43, wherein the sensor system is within the inflation chamber and the inflatable bladder contains a translucent window to allow light to pass therethrough.

Example 45. The blood pressure cuff of any one of examples 36 to 43, wherein the sensor system is within the inflation chamber and the inflatable bladder is translucent to allow light to pass therethrough.

Example 46. The blood pressure cuff of any one of examples 36 to 45, wherein the sensor system is on the inner contact sheet of the inflatable bladder.

Example 47. The blood pressure cuff of any one of examples 36 to 46, wherein the housing comprises a knuckle contour that assists in alignment of the sensor system.

Example 48. A blood pressure monitoring system, comprising:
a blood pressure cuff comprising a housing, an inflatable bladder, and sensor system, the inflatable bladder in connection with the housing such that an inflation chamber is formed in between the inflatable bladder and the housing, wherein the inflatable bladder comprises an elastomeric molded material in a tubular shape, wherein the housing is fixed in a tubular shape, wherein the sensor system comprises a light emitter and light sensor;
a pressure source in fluidic connection with the inflation chamber via a fluid port;
a pressure regulator system provided in between the pressure source and the blood pressure cuff; and
a computational processing system in communication with the sensor system and the pressure regulator system.

Example 49. The system of example 48, wherein the inflatable bladder comprises an inner contact sheet having a tubular shape, a set of angled segments, and two tubular edges; wherein each angled segment is in between the inner contact sheet and one of the tubular edges of the inflatable bladder.

Example 50. The system of example 49, wherein each angled segment has an angle, a density, and a thickness capable of providing a push force, and wherein the thickness of each angled segment is greater than the thickness of the inner contact sheet.

Example 51. The system of example 49 or 50, wherein a vertex is formed between a face of each angled segment and a face of the inner contact sheet; wherein each vertex independently has an angle between 90 degrees and 150 degrees.

Example 52. The system of example 49, 50, or 51, wherein each angled segment has a tapered thickness such that the thickness proximal to the tubular edge is greater than the thickness proximal to the inner contact sheet.

Example 53. The system of any one of examples 49 to 52, wherein the inner contact sheet is composed of flexible and has thickness such that it can expand and contract in accordance with inflation and deflation of the inflation chamber.

Example 54. The system of example 53, wherein the inner contact sheet has a thickness between 25 μm and 100 μm.

Example 55. The system of example any one of examples 49 to 54 further comprising a stabilizer within the inflation chamber.

Example 56. The system of example 55, wherein the stabilizer is a sponge or foam fill.

Example 57. The system of example 56, wherein the sponge or foam fill contains at least 50% open cells.

Example 58. The system of example 56 or 57, wherein the sponge or foam fill is composed of material to yield a viscoelastic sponge or foam.

Example 59. The system of example 56, 57, or 58, wherein the sponge or foam fill has a density gradient.

Example 60. The system of example 59, wherein the sponge or foam fill has greater density near the angled segments of the inflatable bladder and less density near the center of the inflatable bladder.

Example 61. The system of any one of examples 56 to 60, wherein the sponge or foam fill includes one or more windows to allow light signals to pass therethrough.

Example 62. The system of any one of examples 56 to 61, wherein the sponge or foam fill includes one or more apertures, one or more slits, or one or more segments.

Example 63. The system of example 55, wherein the stabilizer is a set of one or more springs.

Example 64. The system of example 63, wherein each spring of the set is a clover-shaped ring.

Example 65. The system of example 64, wherein each clover-shaped ring contacts and is secured to the housing at multiple points, wherein each clover-shaped ring contacts or is adjacent to the inflatable bladder at multiple points, wherein each clover-shaped ring includes an interconnecting portion between each contact with the housing and each contact or adjacency with the inflatable bladder, and wherein each interconnecting portion includes a first hinge near the contact with the housing and a second hinge near the contact or adjacency with the inflatable bladder.

Example 66. The system of example 65, wherein the first hinge and the second hinge each individually have an angle between about 30 degrees and about 150 degrees.

Example 67. The system of example 63, wherein each spring of the set is an elongated ribbon that is elongated in a direction along a tubular axis of the blood pressure cuff.

Example 68. The system of example 67, wherein each elongated ribbon includes a secured end that is secured to the housing, an unsecured end that is free to move in a direction along the tubular axis, and a curved portion between the secured end and the unsecured end.

Example 69. The system of example 68, wherein an apex of the curved portion is in contact with or adjacent to the inflatable bladder.

Example 70. The system of any one of examples 63 to 69, wherein each spring of the set is composed a material with high resiliency or high linear elasticity.

Example 71. The system of example 55, wherein the stabilizer is a set of one or more columnar supports.

Example 72. The system of example 71, wherein each columnar support of the set of columnar supports is in contact with a vertex adjacent to an angled segment of the set of angled segments and further in contact with and secured to the housing.

Example 73. The system of example 72, wherein each columnar support of the set of columnar supports comprises a base, wherein each base provides contact is secured to the housing.

Example 74. The system of example 71, 72, or 73, wherein each columnar support is composed of an elastomeric material.

Example 75. The system of any one of examples 71 to 74, wherein each columnar support of the set of columnar supports is individually provided at an angle from about 45-degrees to about 90-degrees from the housing to the vertex.

Example 76. The system of any one of examples 71 to 75, wherein the set of one or more columnar supports and inflatable bladder are a single mold.

Example 77. The system of any one of examples 48 to 76, wherein the pressure regulator system is capable of sensing and regulating the amount of pressure being provided to the inflation chamber via the pressure source.

Example 78. The system of any one of examples 48 to 77, wherein the sensor system is a photoplethysmograph and capable of transmitting arterial diameter data to the computational system.

Example 79. The system of any one of examples 48 to 78, wherein the housing comprises a knuckle contour that assists in alignment of the sensor system.

Example 80. A molded inflatable bladder for use within a blood pressure cuff, comprising:
an elastomeric molded material in a tubular shape comprising comprises an inner contact sheet having a tubular shape, a first angled segment, a second angled segment, a first tubular end and a second tubular end; wherein the first angled segment is in between the inner contact sheet and the first tubular end; wherein the second angled segment is in between the inner contact sheet and the second tubular end;
wherein a first angled segment angles from the inner contact sheet outward to a first tubular end and a second angled segment angles from the inner contact sheet outward to a second tubular end.

Example 81. The molded inflatable bladder of example 80, wherein each angled segment has an angle, a density, and a thickness for providing a push force, and wherein the thickness of each angled segment is greater than the thickness of the inner contact sheet.

Example 82. The molded inflatable bladder of example 81, wherein a vertex is formed between each angled segment and the inner contact sheet; wherein each vertex independently has an angle between 90 degrees and 150 degrees.

Example 83. The molded inflatable bladder of example 81 or 82, wherein each angled segment has a tapered thickness such that the thickness proximal to the tubular end is greater than the thickness proximal to the inner contact sheet.

Example 84. The molded inflatable bladder of any one of examples 80 to 83, wherein the inner contact sheet is composed of flexible and has thickness such that it can expand and contract.

Example 85. The molded inflatable bladder of example 84, wherein the inner contact sheet has a thickness between 25 μm and 100 μm.

Example 86. The molded inflatable bladder of any one of examples 80 to 85, wherein the elastomeric molded material comprises a means for securement to a housing of a blood pressure cuff, the housing having a tubular shape such that when the elastomeric molded material is secured to the housing, an inflation chamber is formed between the elastomeric molded material and the housing.

Example 87. The molded inflatable bladder of any one of examples 86, wherein the means for securement is a flap that at each tubular end of the elastomeric molded material, and wherein each flap is configured to provide securement via a set of two end caps that are configured to secure the inflatable bladder to the housing via each flap.

Example 88. The molded inflatable bladder of any one of examples 80 to 87, wherein the inner contact sheet, the first tubular end, and the second tubular end are concentric.

Example 89. The molded inflatable bladder of any one of examples 80 to 88 further comprising a set of one or more columnar supports.

Example 90. The molded inflatable bladder of example 89, wherein each columnar support of the set of columnar supports is in contact with a vertex adjacent to an angled segment of the set of angled segments and extends outwardly away from the vertex.

Example 91. The molded inflatable bladder of example 89 or 90, wherein each columnar support of the set of columnar supports is comprises a base, wherein each base provides a means of securement to a housing.

Example 92. The molded inflatable bladder of example 90 or 91, wherein each columnar support of the set of columnar supports is individually provided at an angle from about 45-degrees to about 90-degrees from a housing wall to the vertex.

Example 93. The molded inflatable bladder of any one of examples 89 to 92, wherein the set of one or more columnar supports and the inflatable bladder are a single mold.

Example 94. The molded inflatable bladder of any one of examples 80 to 93 further comprising an outer frame secured to the molded inflatable bladder, wherein an inflation chamber is formed between the outer frame and the molded inflatable bladder; wherein the outer frame and inflatable bladder are configured to be removably installed within a housing such that the housing can be reused after use and removal of the inflatable bladder.

Example 95. A set of components for assembly of a blood pressure cuff, comprising:
a housing fixed in a tubular shape; and
an inflatable bladder comprising an elastomeric molded material in a tubular shape comprising comprises an inner contact sheet having a tubular shape, a first angled segment, a second angled segment, a first tubular end and a second tubular end; wherein the first angled segment is in between the inner contact sheet and the first tubular end; wherein the second angled segment is in between the inner contact sheet and the second tubular ends;
wherein a first angled segment angles from the inner contact sheet outward to a first tubular end and a second angled segment angles from the inner contact sheet outward to a second tubular end;
wherein the inflatable bladder comprises a means for securement to the housing such that when the inflatable bladder is secured to the housing, an inflation chamber is formed between the inflatable bladder and the housing.

Example 96. The set of components of example 95, wherein each angled segment has an angle, a density, and a thickness for providing a push force, and wherein the thickness of each angled segment is greater than the thickness of the inner contact sheet.

Example 97. The set of components of example 96, wherein a vertex is formed between a face of each angled segment and a face of the inner contact sheet; wherein each vertex independently has an angle between 90 degrees and 150 degrees.

Example 98. The set of components of example 96 or 97, wherein each angled segment has a tapered thickness such that the thickness proximal to the tubular end is greater than the thickness proximal to the inner contact sheet.

Example 99. The set of components of any one of example 95 to 98, wherein the inner contact sheet is composed of flexible and has thickness such that it can expand and contract in accordance with inflation and deflation of the inflation chamber.

Example 100. The set of components of example 99, wherein the inner contact sheet has a thickness between 25 μm and 100 μm.

Example 101. The set of components of any one of examples 95 to 100, wherein the housing comprises a fluid port that is configured such that it is in fluidic connection with the inflation chamber when the inflatable bladder is secured to the housing.

Example 102. The set of components of any one of examples 95 to 101, wherein the inner contact sheet, the first tubular end, and the second tubular end are configured to be concentric when assembled.

Example 103. The set of components of any one of examples 95 to 102, wherein the means for securement is a flap that at each tubular end of the inflatable bladder, and wherein each flap is configured to provide securement via a set of two end caps that are configured to secure the inflatable bladder to the housing via each flap.

Example 104. The set of components of any one of examples 95 to 103 further comprising an outer frame secured to the inflatable bladder, wherein an inflation chamber is further formed between the outer frame and the molded inflatable bladder; wherein the outer frame and inflatable bladder are configured to be removably installed within the housing such that the housing can be reused after use and removal of the inflatable bladder.

Example 105. The set of components of any one of examples 95 to 104 further comprising a stabilizer, wherein the stabilizer is configured to fit within the inflation chamber when assembled.

Example 106. The set of components of example 105, wherein the stabilizer is a sponge or foam fill.

Example 107. The set of components of example 105, wherein the stabilizer is a set of one or more springs.

Example 108. The set of components of example 107, wherein each spring of the set is a clover-shaped ring.

Example 109. The set of components of example 107, wherein each spring of the set is an elongated ribbon that is elongated in a direction along a tubular axis of the blood pressure cuff when assembled.

Example 110. The set of components of example 105, wherein the stabilizer is a set of one or more columnar supports.

Example 111. The set of components of example 110, wherein the set of one or more columnar supports and the inflatable bladder are a single mold.

Example 112. The set of components of any one of examples 95 to 111 further comprising a sensor system comprising light emitter and a light sensor configured to be installed within the housing or within the inflatable bladder.

Example 113. The set of components of example 112, wherein the sensor system is a photoplethysmograph.

What is claimed is:

1. A blood pressure cuff for use in a blood pressure measurement system, comprising:
a housing and an inflatable bladder, the inflatable bladder in connection with the housing such that an inflation chamber is formed in between the inflatable bladder and the housing, wherein the inflatable bladder comprises an elastomeric molded material in a tubular shape, and wherein the housing is fixed in a tubular shape;
wherein the inflatable bladder is contoured to conform to an extremity of an individual;
wherein the inflatable bladder comprises an inner contact sheet having a tubular shape, a set of angled segments, and two tubular edges; and
wherein each angled segment is in between the inner contact sheet and one of the tubular edges of the inflatable bladder.

2. The blood pressure cuff of claim 1, wherein each angled segment has an angle, density and a thickness for providing a push force, and wherein the thickness of each angled segment is greater than the thickness of the inner contact sheet.

3. The blood pressure cuff of claim 2, wherein:
a vertex is formed between a face of each angled segment and a face of the inner contact sheet, and wherein each vertex independently has an angle between 90 degrees and 150 degrees; and/or
each angled segment has a tapered thickness such that the thickness proximal to the tubular edge is greater than the thickness proximal to the inner contact sheet.

4. The blood pressure cuff of claim 1, wherein the inner contact sheet is flexible and has a thickness between 25 μm and 100 μm such that it can expand and contract in accordance with inflation and deflation of the inflation chamber.

5. The blood pressure cuff of claim 1, wherein the inflatable bladder is a single molded material comprising the inner contact sheet and the set of angled segments.

6. The blood pressure cuff of claim 1, wherein the inflatable bladder is a welded set of components comprising the inner contact sheet and the set of angled segments.

7. The blood pressure cuff of claim 1, further comprising a set of two end caps, wherein the inflatable bladder further comprises a flap at each tubular end, and wherein the set of two end caps is utilized to secure the inflatable bladder to the housing via each flap.

8. The blood pressure cuff of claim 1, wherein the inflatable bladder comprises a set of two folded-over ends that each fold over an edge of the housing.

9. The blood pressure cuff of claim 1, further comprising an outer frame secured to the inflatable bladder, wherein the inflation chamber is further formed between the outer frame and the inflatable bladder, and wherein the outer frame and the inflatable bladder are configured to be removably installed within the housing such that the housing can be reused after use and removal of the inflatable bladder.

10. The blood pressure cuff of claim 1, wherein the inflatable bladder comprises a set of one or more furrows along at least one vertex between the inner contact sheet and an angled segment of the set of angled segments.

11. The blood pressure cuff of claim 1, further comprising a stabilizer within the inflation chamber.

12. The blood pressure cuff of claim 11, wherein the stabilizer is a sponge or foam fill, and wherein the sponge or foam fill has a density gradient in which the sponge or foam fill has greater density near the angled segments of the inflatable bladder and less density near the center of the inflatable bladder.

13. The blood pressure cuff of claim 11, wherein the stabilizer is a set of one or more springs, wherein each spring of the set of one or more springs is a clover-shaped ring that contacts and is secured to the housing at multiple points, wherein each clover-shaped ring contacts or is adjacent to the inflatable bladder at multiple points, wherein each clover-shaped ring includes an interconnecting portion between each contact with the housing and each contact or adjacency with the inflatable bladder, and wherein each interconnecting portion includes a first hinge near the contact with the housing and a second hinge near the contact or adjacency with the inflatable bladder.

14. The blood pressure cuff of claim 11, wherein the stabilizer is a set of one or more springs, wherein each spring of the set of one or more springs is an elongated ribbon that is elongated in a direction along a tubular axis of the blood pressure cuff, wherein each elongated ribbon includes a secured end that is secured to the housing, an unsecured end that is free to move in a direction along the tubular axis, and a curved portion between the secured end and the unsecured end, and wherein an apex of the curved portion is in contact with or adjacent to the inflatable bladder.

15. The blood pressure cuff of claim 11, wherein the stabilizer is a set of one or more columnar supports, wherein each columnar support of the set of one or more columnar supports is in contact with a vertex adjacent to an angled segment of the set of angled segments and further in contact with and secured to the housing, and wherein the set of one or more columnar supports and the inflatable bladder are a single mold.

16. The blood pressure cuff of claim 1, further comprising a sensor system comprising a light emitter and a light sensor.

17. The blood pressure cuff of claim 16, wherein the sensor system is a photoplethysmograph.

18. The blood pressure cuff of claim 16, wherein the sensor system is within the inflation chamber and the inflatable bladder contains a translucent window to allow light to pass therethrough.

19. The blood pressure cuff of claim 16, wherein the sensor system is within the inflation chamber and the inflatable bladder is translucent to allow light to pass therethrough.

20. The blood pressure cuff of claim 16, wherein the sensor system is on the inner contact sheet of the inflatable bladder.
